(19) United States
(12) Patent Application Publication
(10) Pub. No.: US 2023/0270843 A1
(43) Pub. Date: Aug. 31, 2023

(54) Post-Exposure Vaccination Against Viral Respiratory Infections

(71) Applicants: Jan Ter Meulen, Mercer Island, WA (US); RiboxX GmbH, Dresden (DE); Trenzyme GMBH, Konstanz (DE)

(72) Inventors: Jan Ter Meulen, Mercer Island, WA (US); Jacques Rohayem, Dresden (DE); Reinhold Horlacher, Konstanz (DE)

(21) Appl. No.: 18/005,780

(22) PCT Filed: Jul. 14, 2021
(86) PCT No.: PCT/EP2021/069673

§ 371 Date: Jan. 17, 2023

(30) Foreign Application Priority Data
Jul. 14, 2020 (EP) 20185839.6
Jul. 15, 2020 (EP) 20185962.6

(51) Int. Cl. A61K 39/215; A61K 39/145; A61K 39/155; A61K 39/12; A61K 39/285; A61K 39/39; A61P 31/14; A61P 31/16

(52) U.S. Cl. CPC A61K 39/215; A61K 39/145; A61K 39/155; A61K 39/12; A61K 39/285; A61K 39/39; A61P 31/14; A61P 31/16; A61K 2039/543

(57) ABSTRACT

Pharmaceutical compositions, in particular vaccine compositions, for preventing or at least reducing the severity of, respectively, viral respiratory infections through application of said composition to a human subject post-exposure or at least presumed post-exposure of said subject to a virus causing said viral respiratory infections or pre-exposure of said subject to said virus. More particularly, in specific embodiments, the invention provides pharmaceutical compositions as such comprising at least one antigenic component of the infectious virus and a TLR-3 agonist. The invention also relates to methods of treatment and/or prevention of said viral respiratory infections through administration of the composition to the human subject post exposure or at least presumed post-exposure of said subject to the infectious virus or pre-exposure of said subject to said virus

### FIELD OF THE INVENTION

The present invention relates to pharmaceutical compositions, in particular vaccine compositions, for preventing or at least reducing the severity of, respectively, viral respiratory infections through application of said composition to a human subject post-exposure or at least presumed post-exposure of said subject to a virus causing said viral respiratory infections or pre-exposure of said subject to said virus. More particularly, in specific embodiments, the invention provides pharmaceutical compositions as such comprising at least one antigenic component of the infectious virus and a TLR-3 agonist. The invention also relates to methods of treatment and/or prevention of said viral respiratory infections through administration of the composition to the human subject post exposure or at least presumed post-exposure of said subject to the infectious virus or pre-exposure of said subject to said virus.

### REFERENCE TO SEQUENCE LISTING

This application contains a Sequence Listing. The application thus incorporates by reference the material in the ASCII text file 04944P0005ASeqList.txt, created on Jan. 16, 2023, and having a size of 92,508 bytes.

### BACKGROUND OF THE INVENTION

Pandemic respiratory infections caused by SARS-like coronaviruses (SARS-CoV1 and SARS-CoV2) and influenza virus (avian H5N1 and swine H2N1 and H1H1 influenza) have occurred roughly every 10 years in the past half century with tens of millions of people infected and hundreds of thousands having succumbed to these diseases (Monto 2020, Gates 2020). The current SARS-CoV2 pandemic is still ongoing and the disease will likely become endemic and seasonal, comparable to influenza. Both virus families exist in nature in large animal reservoirs, birds and bats respectively, and spill-over to the human population is assumed to intensify in the coming decades with ongoing human population growth and habitat destruction. Natural immunity to either virus, if it develops, is typically specific to the infecting strain and with SARS-like coronaviruses likely not very durable. While vaccines against SARS-like viruses are currently not available, pandemic influenza vaccines have been successfully manufactured and in the case of the 2009 H1N1 pandemic virus been used in mass immunization campaigns. While influenza vaccines prevent disease, systemic administration of killed influenza vaccines or of high-titred anti-influenza IgG antibodies has been shown ineffective in protecting vaccinated guineapigs from becoming infected with influenza virus by transmission from an infected cage mate (Lowen 2009). In contrast, induction of local and systemic IgA antibodies with live attenuated influenza vaccines or transfer of systemic IgA antibodies was protective against infection and disease (Seibert 2013). Live attenuated influenza vaccines have also been shown to reduce viral shedding in pigs by approx. 50% (Kaiser 2018).

In the absence of prophylactic vaccines against pandemic influenza viruses, the development of which requires identification and cloning of the RNA of the infecting viruses during a pandemic, there are no pharmacological interventions available which could both prevent disease in infected patients and stop the patient from transmitting the viruses. Antiviral drugs with activity against influenza virus, such as neuraminidase inhibitors, have shown limited efficacy against avian influenza strains and in a post-exposure setting can lead to rapid generation of transmissible resistant virus strains. Therefore, strategies have been recommended that prioritize the treatment of only ill individuals, rather than the prophylaxis of those suspected of being exposed, as most effective in reducing the morbidity and mortality of the pandemic (Moghadas 2009). This will obviously only modestly impact the spread of the virus as the pool of susceptible persons is not reduced and if infected, they continue to transmit the virus.

The availability of a post-exposure treatment of persons exposed to SARS coronaviruses, pandemic influenza viruses, or both, which could prevent infection, disease and transmission would therefore be a game changer in the containment of ongoing and future pandemics. Ideally, such a treatment should be readily available and self-administered to circumvent logistic problems arising from health care resources typically being focused on treating sick patients during epidemics.

SARS-CoV-2 is an emerging coronavirus likely originating from bats in China which is easily transmitted via the respiratory route and currently causing a pandemic in the human population. The multifaceted disease with respiratory and systemic pathology caused by SARS-CoV-2 in humans is named COVID-19. While 80% of infected persons experience no or only mild upper or lower respiratory symptoms and fever, 20% of COVID-19 patients develop severe disease requiring medical attention or hospitalization. The case-fatality-rate of the disease is high and age-dependent, ranging from 1-3% for all infections, but may be as high as 20% in the elderly with pre-existing medical conditions (Tay 2020, Long 2020). The FDA has issued an emergency use authorization for the antiviral to treat COVID-19, Remdesivir, a RNA-dependent RNA polymerase inhibitor which showed limited efficacy in clinical trials. No vaccine is currently available to prevent or treat COVID-19, and no vaccine has been developed for any other human coronavirus.

SARS-CoV-2 is highly transmissible by droplets generated mainly in the upper respiratory tract through sneezing, coughing, singing, and speaking, but possibly also by breathing. Aerosol transmission is likely possible in small, crowded, poorly ventilated spaces (Li 2020). In the majority of cases viral infection is initially established in the nasopharyngeal tract, from where the virus is shed in pre-symptomatic patients up to 2 days prior to development of symptoms, when infectivity peaks (Hou 2020, Wang 2020). The magnitude of the viral load measured by RT/PCR in nasopharyngeal swabs is a predictor of mortality (Pujadas 2020). Pre-symptomatic virus transmission may account for as much 40% of all infections and constitutes a major public health concern with respect to controlling the pandemic (He 2020). No pharmaceutical means are currently available to protect from infection after exposure or to prevent human-to-human transmission of the virus, and therefore (self)quarantine (lockdown) and wearing of facemasks are the principal interventions by which outbreaks need to be controlled. Lockdown has a profound impact on the economic activity of affected regions and countries.

SARS-CoV-2 is a positive-sense single-stranded RNA virus belonging to the family of beta-coronaviruses; the diameter of the virus particles ranges from 60 to 140 nm with distinctive “spikes” about 8 to 12 nm in length. The viral genome of SARS-CoV-2 is around 29.8 kilobase, with a G+C content of 38%, in total consisting of six major open reading frames (ORFs) common to coronaviruses and a number of other accessory genes. The spike (S) protein of beta-coronaviruses is expressed on the virion surface as a transmembrane homotrimer, with proteolytic cleavage yielding S1 and S2 subunits. S mediates both recognition of cellular receptor(s) and membrane fusion. In the case of SARS-CoV-1 and SARS-CoV-2, which share 80% amino acid sequence identity in the spike protein, a receptor binding domain (RBD, amino acids 318-510) within S1 directly interacts with high affinity with the peptidase domain of angiotensin-converting enzyme 2 (ACE2). The S2 subunit of S mediates membrane fusion and is cleaved from the S1 subunit by trypsin-like proteases (cathepsin, TMPRSS2) or furin as part of the maturation process of the virus resulting in infectivity (Jaimes 2020).

The S/ACE2 interaction mediates viral entry and provides an attractive target for vaccine-elicited humoral immunity with antibodies potentially capable of either (i) directly blocking binding of ACE2 by (ii) blocking conformational changes in S critical for membrane fusion, (iii) eliminating infected cells through antibody effector mechanisms such as antibody dependent cellular cytotoxicity (ADCC), or (iv) driving accelerated clearance of free virus, Most convalescent plasmas obtained from individuals who recover from COVID-19 do not contain high levels of SARS-CoV2 neutralizing activity, however, potent neutralizing monoclonal antibodies (mAb) have been isolated from many patients and it was shown that the vast majority of theses are directed against the RBD, which make it an attractive target for vaccine development (Robbiani 2020, Zost 2020, Rogers 2020). Further epitope mapping revealed that mAbs can neutralize either by direct binding to the ACE2 docking site on the RBD, thereby blocking interaction with the receptor, or by allosteric mechanisms through binding to the RBD outside of this site. We have previously described human mAb CR3022, recovered from a SARS-CoV-1 patient, that reacts with the cryptic epitope in the RBD of SARS-CoV-1 and SARS-CoV-2, which is 100% conserved, and neutralizes both viruses. Combining CR3022 with the SARS-CoV-1 neutralizing mAb CR3014, which inhibits binding of the RBD to ACE2, was strongly synergistic and made the generation of escape mutants in cell culture impossible (ter Meulen 2006). Binding of mAbs CR3022 and CR3014 (or recombinant ACE2) are therefore important assay to characterize the structural integrity of recombinantly expressed SARS-CoV2 as a vaccine antigen.

SARS-CoV2 infection is able to suppress innate and adaptive immune responses on multiple levels. In infected cell lines, primary bronchial cells, and a ferret model a lack of robust type I/III Interferon (IFN) signatures was noted (Blanco-Melo et al., 2020). Patients with severe COVID-19 demonstrate impaired IFN type I signatures as compared to mild or moderate cases (Hadjadj 2020) and defects in the number and/quality of NK cells and T-cells have also been observed and reported to correlate with the severity of clinical disease (Reviewed in Vabret 2020).

In COVID patients, virus-specific IgM and IgG are detectable in serum between 7 and 14 days after the onset of symptoms. Viral RNA is inversely correlated with neutralizing antibody titers. Higher titers have been observed in critically ill patients, but it is unknown whether antibody responses somehow contribute to pulmonary pathology. The SARS-CoV-1 humoral response is relatively short lived, and memory B cells may disappear, suggesting that immunity with SARS-CoV-2 may wane 1-2 years after primary infection (reviewed in Vabret 2020).

A recent study in rhesus macaques, which to a certain extent replicate human lung pathology when challenged with the virus, but not severe disease, demonstrated humoral and cellular immune responses and protection against re-challenge. Residual low levels of subgenomic viral mRNA in nasal swabs in a subset of animals and anamnestic immune responses in all animals following SARS-CoV-2 rechallenge suggested that protection was mediated by immunologic control and likely was not sterilizing (van Doremalen 2020). In addition, persistent shedding of SARS-CoV2 in feces and nasopharyngeal secretions has been observed in COVID patients, in the latter case of up to 49 days after onset of disease (Tan 2020).

Several vaccines for SARS-CoV-1 were developed and tested in animal models, including recombinant S-protein-based vaccines, attenuated and whole inactivated vaccines, and vectored vaccines (reviewed in Roper 2009). Most of these vaccines protected animals from challenge with SARS-CoV-1, although most did not induce sterilizing immunity. In some cases, vaccination with especially inactivated whole virus vaccines containing aluminum adjuvants resulted in complications, including lung damage and infiltration of eosinophils in a mouse model (Bolles 2011, Tseng 2012) and liver damage in ferrets (Weingartl 2004). In another study, vaccination with inactivated SARS-CoV-1 led to enhancement of disease in one NHP, whereas it protected 3 animals from challenge (Wang 2016). The same study identified certain epitopes on the S protein as protective, whereas immunity to others seemed to be enhancing disease. However, in almost all cases, vaccination is associated with greater survival, reduced virus titers, and/or less morbidity compared with that in unvaccinated animals. A large number (>140) of SARS-CoV-2 vaccines are currently mainly in preclinical development, based on a multitude of different platforms, such as inactivated whole virus, DNA or mRNA constructs expressing the spike protein (often in a prefusion-stabilized conformation), different recombinant viral vectors expressing the spike protein (adenoviral vectors, measles vectors and others) and many others. The vast majority of these approaches involves intramuscular or subcutaneous injection of the vaccine, which results in induction of systemic IgM and IgG antibodies that act to prevent pneumonia and other severe systemic disease but will not induce secretory IgA antibodies that can prevent infection and viral shedding in the nasopharynx. As a case in point, a SARS-CoV-2 spike vaccine based on a vector derived from a chimpanzee adenovirus that recently entered phase 2 testing, was shown to prevent COVID-19 pneumonia in rhesus monkeys, but it neither prevented infection nor reduced viral titers in the nasopharynx compared to unvaccinated controls (van Doremalen 2020).

Based on the mechanism of action of the above-mentioned vaccines there is little indication that any of them will be (i) effective in the post-exposure setting, because of the short incubation period of SARS-CoV2 of approx. 5 days (+/−1, 95% confidence interval), or (ii) will be able to completely prevent transmission of the virus from a vaccinated infected individual to an unvaccinated individual. This is a very undesirable situation in many settings where vaccinated persons come in contact with an unvaccinated vulnerable population, e.g. nursing home residents, and can unknowingly spread the disease. In addition, all injectable vaccines need to be administered by a qualified health care professional and cannot be self-administered.

Mucosal associated lymphoid tissues (MALTs) are important sites for the induction of antigen-specific secretory IgA antibodies (Kiyono 2015). Examples of MALTs include gut-associated lymphoid tissue (GALT) in the intestinal tract and nasopharynx-associated lymphoid tissue (NALT) in the respiratory tract. MALT contains lymphocytes, M cells, T cells, B cells and antigen-presenting cells (APCs), and the efficient delivery of antigens into MALT is essential for mucosal vaccinations (Kunisawa 2008). Antigens that contact the epithelial surface of GALT and NALT are taken up by M cells located in areas called the follicle-associated epithelium (Kanaya 2012, Sato 2013). After uptake, the antigens are delivered to antigen-presenting cells such as dendritic cells (Kunisawa 2012). The antigen-presenting cells then process the antigens into peptides and transport them to naïve helper T cells, which primes the helper T cells (Kelsall 1996). The antigen-primed helper T cells support the induction of somatic hypermutation by B cells and immunoglobulin class switching in germinal centers (Mora 2006). Therefore, MALTs are considered good target for mucosal vaccine antigens to induce antigen-specific immune responses and there have been several attempts in the past to deliver antigens to MALT using microparticles, liposomes, saponins or chitosans (Manocha 2005).

Claudin-4, which in humans is encoded by the CLDN4 gene, is widely expressed on NALT and has been identified as a candidate M cell endocytosis receptor (Lo 2004, Kakutani 2010, Wang 2009). The C-terminus of C. perfringens enterotoxin (C-CPE) is a receptor-binding fragment selectively targeting claudin 4 and its C-terminal amino acid 194-319 fragment has high solubility and affinity and is capable of enhancing mucosal absorption of drugs, while having low antigenicity itself (reviewed in Lan 2019). Moreover, C-CPE showed no cytotoxicity to cells expressing claudin-4 in vitro and in mice nasal administration of C-CPE caused no mucosal injury in the nasal cavity or nasal passages. Therefore, C-CPE has potential as a claudin-4-targeting antigen tag and has been used to increase the immunogenicity of intranasal pneumococcal and influenza vaccines (Suzuki 2015, Lo 2012). Both vaccines induced increased production of IgA in nasopharyngeal secretions and bronchio-alveolar lavage. Interestingly, the M cell targeting influenza vaccine led to increased production of mucosal and serum IgA, but not systemic IgG antibodies against HA.

Vaccines are preparations of antigenic materials, administered to recipients with a view to enhancing resistance to infection by inducing active immunity to specific microorganisms, for example viruses. Vaccines, which may be single or mixed component vaccines, are presented in a variety of forms and typically administered to a recipient prior to exposure to the infectious agent, as so-called prophylactic vaccines. Vaccines are used prophylactically because it usually requires approx. 10 days to develop protective levels of antibodies and T-cells in 90% of recipients, even with potent vaccines, e.g. live attenuated yellow fever virus (Monath 2001). In some infectious diseases that are known to have long incubation times of weeks to months, e.g. rabies and viral hepatitis, post-exposure prophylaxis with vaccines is standard medical practice, and also in diseases with incubation periods of between 10 and 14 days (e.g. measles, mumps, varicella and Variola major) post-exposure vaccination can modify the clinical course to a certain extent (Gallagher 2019). However, in the case of viral diseases with shorter incubation periods, such as SARS coronavirus (median incubation period 4-5 days) or influenza virus infection (incubation period 1-4 days), post-exposure vaccination has been hitherto largely unsuccessful (Gallagher 2019), because the speed of viral replication outpaces the ability of the adaptive immune system to generate antiviral antibodies and T-cells to curb the infection. Furthermore, systemically administered vaccines do not generate immediate or long-term immune responses at the site of viral entry and initial replication, which is the mucosa of the nasopharyngeal tract.

In order to provide protection at the entry site of respiratory viral infections, pre- and post-exposure prophylaxis has been attempted through repeated unspecific stimulation of the NALT using intranasal monotherapy with recombinantly expressed type 1 interferon or agonists of toll-like receptor 3 (poly-lysine/carboxymethyl-cellulose-stabilized polyinosinic-polycytidylic acid, poly-IC:LC) These treatments have resulted in secretion of cytokines in the nasal mucosa particular IP-10 (CXCL-10), TNF-alpha, IL-6 but been moderately effective clinically, reducing symptoms by 30-50%, without reducing shedding (Treanor 1987, Malcolm 2018). There is no published evidence that these treatments induced or accelerated the development of adaptive immune responses against the infectious agents, in particular activation of antigen presenting cells such as dendritic cells, monocytes, as well as activation of T helper CD4+ cells and cytotoxic T cells (CD8+) and Natural Killer cells (NK cells) directed against the virus and/or its virus proteins and antigens, and capable of mounting a specific and potent immune response protective from re-infection with the virus of interest. In a preclinical model repeated prophylactic intranasal application of poly-IC:LC as a monotherapy protected against lethal SARS-CoV1 infection, and reduction of mortality was also observed in a post-exposure setting when given 8 hours after challenge (Koumaki Vaccine 2016).

Currently there is only one approved intranasal vaccine, which is a live attenuated influenza virus vaccine (Flumist®) which is only effective in children, because of the absence of pre-existing immune responses that neutralize the virus (Shannon 2020). Intranasal immunization of humans with inactivated influenza virus vaccine has been reported in the context of influenza virosomes adjuvanted with enzymatically

inactivated E. coli labile toxin (LTK63), which caused an unacceptable rate of late occurring side effects (Bell's palsy), likely due to the inflammatory nature of the adjuvant (Mutsch 2004, Lewis 2009). Intranasal administration of a toll-like receptor 4 (CRX-601, GSK) combined with detergent split-influenza antigen (A/Uruguay/716/2007 (H3N2) generated strong local and systemic immunity (IgA, IgG antibodies, Th17 T-cells) against co-administered influenza antigens and exhibited high efficacy against heterotypic influenza challenges. However, following challenge with influenza virus, vaccinated mice transiently exhibited increased weight loss and morbidity during early stages of disease due to expansion of vaccine-primed Th17 cells, accompanied by an augmented lung neutrophilic response (Maroof 2014). In mice, intranasal immunization with influenza subunit vaccines adjuvanted with the TLR3 agonist poly-IC/LC was shown to induce IgA in nasal washes and IgG in serum, and the effect was linked to stimulation of CD103 positive mucosal dendritic cells (Takaki 2017). The effectiveness in cynomolgus macaques of intranasal administration of an influenza A H5N1 pre-pandemic vaccine combined with synthetic double-stranded RNA (polyl/polyC12U) as an adjuvant was examined. The monkeys were immunized with the adjuvant-combined vaccine on weeks 0, 3, and 5, and challenged with the homologous virus 2 weeks after the third immunization. After the second immunization, the immunization induced vaccine-specific salivary IgA and serum IgG antibodies, as detected by ELISA. The serum IgG antibodies present 2 weeks after the third immunization not only had high neutralizing activity against the homologous virus, they also neutralized significantly heterologous influenza A H5N1 viruses. The vaccinated animals were protected completely from the challenge infection with the homologous virus. These results suggest that intranasal immunization with the double stranded RNA-combined influenza A H5N1 vaccine induced mucosal IgA and serum IgG antibodies which could protect humans from homologous influenza A H5N1 viruses which have a pandemic potential. In non-human primates, a pre-pandemic H5 virus vaccine (whole inactivated) adjuvanted with a TLR3 agonist (polyl:polyC12U, Rintatolimod) induced antiviral secretory IgA and systemic IgG antibodies. In humans, application of Rintatolimod several times intranasally after intranasal immunization with trivalent attenuated influenza vaccine (Flumist®) increased homologous secretory IgA titers at least 4-fold and induced antibodies cross-reactive with three clades of hemagglutinin (Overton 2014, Ichinohe 2010).

Intranasal vaccines for SARS-like coronaviruses (SARS-CoV1, MERS) have been developed preclinically based on adenoviral, adeno-associated virus, Newcastle disease virus and parainfluenza virus vector platforms, recombinant virus like particles adjuvanted with CpG, recombinant RBD as a fusion protein with the Fc part of human IgG and other approaches The vaccines induced systemic virus-specific neutralizing antibodies and T-cell responses comparable to systemic vaccination, but significantly higher local mucosal immune responses (Jia 2019, Kim 2019, DiNapoli 2007, Du 2008, Lu 2009, Ma 2014, Li 2020).

Intranasal vaccines for SARS-like coronaviruses (SARS-CoV1, MERS) have been developed preclinically based on adenoviral, adeno-associated virus, Newcastle disease virus and parainfluenza virus vector platforms, recombinant virus like particles adjuvanted with CpG, recombinant RBD as a fusion protein with the Fc part of human IgG and other approaches The vaccines induced systemic virus-specific neutralizing antibodies and T-cell responses comparable to systemic vaccination, but significantly higher local mucosal immune responses (Jia 2019, Kim 2019, DiNapoli 2007, Du 2008, Lu 2009, Ma 2014, Li 2020).

In one aspect, the technical problem underlying the present invention is the provision of a safe and effective therapeutic means for protecting human subjects against viral disease development after or before, respectively, exposure of the subject to a virus causing respiratory infection, and preventing transmission through shedding of the said virus.

### SUMMARY OF THE INVENTION

The invention relates to methods for generation of both immediate innate immune responses and long-term adaptive immune responses against viral respiratory infections by administration of a pharmaceutical composition, hereinafter also referred to as a vaccine, comprising a combination of one or more antigenic components of the virus, such as viral proteins or parts thereof, and one or more adjuvants eliciting an innate immune response in the subject.

In preferred embodiments of the invention, the combination is formulated as a post-exposure intra-nasal vaccine against airborne transmitted viruses such as SARS-CoV2 and genotype 4 (G4) Eurasian avian-like (EA) H1N1 influenza virus containing the recombinantly expressed Spike protein, preferably only the Spike receptor binding domain (RBD) of SARS-CoV2 and/or the recombinantly expressed head or stem region of the H1 influenza virus hemagglutinin, administered as vaccine antigens alone or as fusion proteins with Claudin-4 targeted domains, preferably as a fustion construct with C-CPE, adjuvanted with a synthetic toll-like receptor agonist, preferentially a TLR-3 agonist.

The invention also provides methods for the formulation of vaccines according to the invention and a pharmaceutical kit comprising an intranasal administration device and a vaccine as described herein.

The present invention is particularly directed to the use of pharmaceutical compositions for prevention and/or treatment of a viral respiratory infection in a subject wherein said composition comprises (i) at least one antigenic component of the virus causing the respiratory infection and/or a nucleic acid, preferably an mRNA, encoding at least one antigenic peptide component, preferably an epitope of a viral protein, and (ii) one or more adjuvants eliciting an innate immune response in the subject against said virus.

The inventive pharmaceutical composition may be administered to the subject as a prophylactic treatment, i.e. the pharmaceutical composition is administered to the subject before the subject has been exposed to the virus (pre-exposure treatment).

In other embodiments, especially in cases where the infectious virus causes conditions, in particular virus diseases, within a comparatively short incubation time, the pharmaceutical composition is administered to the subject after exposure of the subject to the infectious virus (post-exposure). It is to be understood that it is sometimes or often not straightforward to know with reasonable certainty whether or not the subject has factually been exposed to the virus. Therefore, the inventive treatment (including preventive treatment) is also directed to cases where the subject is presumed or at least suspected to have been exposed to the virus, e.g. if the subject had contact to an infected subject such as a contact within a distance and time period where it is typically known that an exposure of the non-infected subject to the virus is likely to occur or at least there is a certain probability such as about 10% or more, preferably about 20% more, more preferably about 10% or more, preferably 20% or more, more preferably 50% or more that the non-infected subject has been exposed to the virus.

In principle the pharmaceutical composition can be administered by any suitable route that will allow generation of the desired immune response (both innate and long-term). For example, the administration can be by injection, either systemically, e.g. by intramuscular, subcutaneous or intradermal injection, or topically, e.g. transdermally which is typically effected by using a microneedle patch. More preferred administration of the pharmaceutical composition is topically to the respiratory tract, preferably the upper respiratory tract, such as intra-orally or, more preferably, intra-nasally or intrapulmonary.

The invention is particularly directed to treatments of subjects by self-administration, in particular in view of the desired protection against viral respiratory infection in the setting of an outbreak, epidemic or pandemic which are typically caused by diseases having comparatively short incubation times such as 10 days or below. The above-described administration routes to the upper respiratory tract, in particular intra-nasal administration, are highly suitable, and preferred, for self-administration by the subject to be treated according to the invention.

In comparison to prior art approaches, in particular when compared to administration by injection, intra-nasal administration of the pharmaceutical composition has the further benefit of largely avoiding the risk of immune enhancement complications, since intra-nasal application has limited immediate systemic effects.

Especially in the post-exposure (or post-suspected exposure) treatment according to the invention, it is desirable that the composition is administered within a short time after exposure or suspected exposure to the virus (hereinafter referred to as the “first administration”). Preferably, the composition is administered to the subject, preferably to the upper respiratory tract, more preferably intra-nasal, even more preferred by self-administration, within from about several seconds or minutes such as 5 or 10 min, preferably from about 30 min, more preferred within about 1 hour to about 12 hours, preferably to about 1 day, or to about 2 days, or to about 3 days after exposure or suspected exposure to the virus. In preferred embodiments, the first administration comprises the administration of the composition within about 30 seconds to about 72 hours, more preferably within about 3 min to about 24 hours, most preferred within about 5 min to about 12 hours post-exposure (or presumed or suspected exposure) to the subject, preferably to the upper respiratory tract of the subject, more preferably by intra-nasal administration. According to the invention, the above time periods until the first administration takes place, may also be time limits for post-exposure of the human subject after the subject has been diagnosed positive for the presence of the respective virus. This aspect is especially important for clinical settings where suspected patients are tested for the virus, e.g. SARS-CoV-2, and a fast protective treatment is desirable.

The inventive treatment is suitable for any human subjects. However, depending on the specific viral respiratory infections, certain preferred human populations are preferred to benefit from the invention. For example, elder subjects such as of an age of about 50 year or more, preferably about 60 or more, more preferred about 70 or more, are preferably treated for prevention and/or treatment of infection by respiratory viruses, in particular influenza viruses and/or SARV-like coronavirus, with SARS-CoV-2 infection being particularly preferred. Also preferred subjects for the inventive treatments are humans having pre-existing medical conditions or preconditions such as chronic diseases, immune suppression, or other complications, in particular obesity, pre-diabetic conditions, diabetes, in particular diabetes mellitus type 2, and chronic heart and kidney disease. It is evident, that elder subjects, such as of the age ranges as outlined above, having a complication, for example as stated above, will particularly benefit from the inventive treatment.

In preferred embodiments, the above first administration is followed by one or more administration cycles such as, for example an administration period of one, two or three weeks, with one week being especially preferred. In one preferred embodiment, the pharmaceutical composition is administered once about every 48 h for about one week, most preferably once about every 72 h for one week. In other embodiments, the pharmaceutical composition for use in the present invention is administered at least once daily, preferably for one week.

After the above further administration cycle(s), the treatment regimen of the invention preferably comprises at least one further administration to the subject, preferably at least once one week after the above further administration cycle(s),

In the context of the above administration regimen, including each independent administration, the pharmaceutical composition, in particular the vaccine, according to the invention is administered in one or more unit dosages, most preferably in one unit dose, e.g. as outlined for preferred compositions herein below.

The inventive treatment by use of the pharmaceutical composition, in particular the vaccine, as described herein is especially designed for treatment of respiratory viral infections leading to conditions or disease states having a comparatively short incubation time. A “short incubation time” means according to the invention an incubation time of not more than 10 days, preferably at most 9 days, more preferably at most 8 days. It is to be understood that an incubation time as disclosed herein is a statistical value typically representing a mean value with a standard deviation. In preferred embodiments, the incubation time is a mean value+/−standard deviation in a confidence interval of at least about 90%, preferably of at least about 95%, most preferably of at least about 99%. Preferably, the incubation time is about 8 days+/−2 days (95% confidence interval), most preferably about 5+/−1 days (95% confidence interval), or, in other preferred embodiments, most preferably about 3+/−2 days (95% confidence interval).

As already outlined above, the inventive treatment concerns prevention and/or therapy of viral respiratory infections in humans. A “viral respiratory infection” according to the invention is an infection by a virus in the respiratory tract of a human subject. While the viral respiratory infection at least has its origin in the respiratory tract, including the upper respiratory tract (nose and/or mouth and/or naso-pharynx and/or pharynx and/or larynx) and the lower respiratory tract (trachea and/or primary bronchi and/or lungs), i.e. the virus typically enters the human subject via the respiratory tract and infects cells of the respiratory tract of the human subjects, the skilled person is aware that the viral respiratory infection is not to be equated by the diseases states of the human subject caused by said infection, which diseases may or may not include respiratory conditions such as fever, ache of limbs and bones, headache, gastro-intestinal conditions, vomiting and the like.

The present invention is particularly directed to the treatment and/or prevention of viral respiratory infection by SARS-like coronaviruses, preferably SARS-Coronavirus (SARS-CoV) and/or MERS and/or SARS-Coronavirus 2 (SARS-CoV-2), influenza viruses, preferably genotype 4 (G4) Eurasian avian-like (EA) H1N1 influenza virus or an amino acid consensus sequence based on the G4 EA virus, H5N1 and/or other H1N1 and/or H2N2 and/or H3N2 and/or H7N1 viruses, or viruses of the Paramyxoviridae family, preferably Respiratory Syncytial Virus, and/or Parainfluenza Virus 1 and/or Parainfluenza Virus 2 and/or Parainfluenza Virus 3 and/or Hendra virus and/or Nipah Virus, viruses of the Pneumoviridae family, preferably Metapneumovirus and viruses of the Poxviridae family, preferably of Variola major (smallpox) and/or monkeypox.

Highly preferred viral infections to which the inventive treatment is directed are infections by SARS-like coronaviruses, most preferred SARS-CoV-2, and influenza viruses, most preferably H5N1 or H1N1 virus, preferably H5N1 or H1N1 virus subtypes with human epidemic/pandemic potential.

The present invention has the special benefit of combining (i) an innate immune response generated by the one or more adjuvant(s) leading to an immediate protection against the virus by activating inter alia natural killer cells, which is particularly important after exposure or at least after presumed exposure of the subject to the virus, and (i) a long-term protection against the virus by stimulating virus-specific antibodies and T-killer cells, especially in the mucosa of the respiratory tract. The combined strategy of the invention thus reduces, preferably eliminates, shedding of the targeted virus.

The combined strategy of the invention is especially useful in the treatment and/or prevention of coronavirus infections, preferably infection by SARS-like coronavirus, most preferably by SARS-CoV-2, the virus leading to the disease COVID-19.

In particular as regards SARS-CoV-2 infections, the present invention provides a solution to the problem that conventional vaccines for treatment or prevention of COVID-19 leave a protection gap of around 14 days, which is the time period until protective antibodies (IgG) against the virus are measurable (see FIG. 1).

The antigenic component (i.e. one or more thereof) may be any component of a virus as described herein that can elicit an immune response in the subject. Preferred antigenic components are, for example, inactivated virus, virus subunits, viral proteins, including viral structural proteins, virus non-structural proteins, viral enzymes, and peptides comprising an epitope (i.e. one or more) of a protein of said virus. If present in the compositions of the invention, the nucleic acid, preferably an mRNA, encodes an antigenic peptide component which may be a viral protein such as a viral structural protein, a viral non-structural protein, a viral enzyme, and/or a peptide comprising an epitope (i.e. one or more) of a protein of said virus. With respect to antigenic peptide-encoding nucleic acid, preferably mRNA, components which may be additionally or alternatively, respectively, be present in the inventive compositions, uses thereof and methods of treatments employing such compositions, it is to be understood according to the invention that any reference to a peptide component of a virus as described or defined herein, respectively, also includes and refers to, respectively, a corresponding nucleic acid, preferably mRNA, encoding such a peptide component (be it a protein, a protein fragment, domain or peptide epitope).

In the case of prevention and/or treatment of infections by SARS-like coronaviruses, the pharmaceutical composition preferably contains a peptide comprising an epitope of the spike protein of said SARS-like coronavirus, more preferred a peptide comprising an epitope of the receptor binding domain (RBD) of said spike protein and/or a nucleic acid, preferably an mRNA, encoding such a peptide. Preferably, a peptide and/or nucleic acid encoding such a peptide for use in the invention comprises the ACE2-epitope of the SARS-CoV-2 RBD. Other preferred embodiments of peptides for use in the invention comprise the CR3022 epitope of the SARS-CoV-2 RBD. The latter epitope has two benefits: (A) it is 100% conserved in SARS-CoV, in particular SARS-CoV-2, and (B) the epitope is presumably essential to the stability of the spike protein such that no immune escape of the virus from neutralizing antibodies is possible (Ter Meulen 2006). The peptide comprising said epitope (one or more thereof) may include the complete spike protein and/or the RBD or a fragment of said spike protein (as exemplified by the RBD) or of the RBD. Most preferred, the composition comprises the spike protein or a fragment thereof, more preferably the RBD or a fragment thereof, or one or more peptides comprising at least one epitope of said spike protein or RDB of SARS-CoV-2. The terms “SARS-CoV-2 spike protein” and “SARS-CoV-2 RBD” refer to the SARS-CoV-2 wildtype Wuhan-Hu-1 strain as well as any variant of SARS-CoV-2, preferably including, but not limited to variants B.1.18 (also denoted a variant Alpha), B.1.617 (also denoted as variant Delta), variant B.1.351 (also denoted as variant Beta), variant B1.1.28 and variant P.1 (also denoted as variant Gamma) The afore-mentioned SARS-CoV-2 variants are also known as “Variants of Concern” (VOC), and variants Delta and Beta are denoted hereinafter also as UK VOC and SA VOC, respectively.

In the case of influenza viruses to which the inventive treatment is directed according to preferred embodiments, the composition preferably comprises hemagglutinin A1 and/or hemagglutinin A2 or a fragment or a peptide comprising at least one epitope thereof. Preferred hemagglutinin fragments are stem or head fragments, and preferred peptides for use of antigenic components comprise at least one epitope of the stem and/or head fragments of a hemagglutinin, preferably of the above hemagglutinin A1 and/or A2. In preferred embodiments of the invention, the antigenic component of influenza viruses the protein or at least a peptide of said protein has an amino acid consensus sequence based on the genotype 4 (G4) Eurasian avian-like (EA) H1N1 influenza virus (also denoted herein as “G4 EA virus”).

Especially in the context of intra-nasal administration of the pharmaceutical composition, it is preferred to include the antigenic component of the virus, preferably a protein or a peptide comprising an epitope thereof, such as a peptide comprising at least one epitope of a spike protein or a fragment thereof such as an RBD of a SARS-like coronavirus, preferably a peptide comprising at least one epitope of the RBD (including the RBD itself or a fragment thereof) of SARS-CoV-2, or, in the case of influenza vaccination according to the invention, a peptide comprising at least one epitope of a hemagglutinin such as hemagglutinin A1 and/or A2, in a construct comprising a mucosa-targeting moiety. The antigenic component may be coupled or linked to the mucosa-targeting moiety by covalent or non-covalent binding. In preferred embodiments, a protein or peptide, as defined herein-before, is typically covalently linked to the mucosa-targeting moiety which is itself typically a protein or peptide such as an oligo- or polypeptide. Proteins or peptides for use in the invention as antigenic components are typically covalently linked to a mucosa-targeting protein (or peptide, oligopeptide or polypeptide) by recombinant expression of a nucleic acid coding for said construct, typically comprised in a suitable expression vector, in a host cell. A preferred mucosa-targeting moiety for use in the invention is the C-terminal fragment of Clostridium perfringens Enterotoxin (C-CPE). In highly preferred embodiments of the invention, the C-CPE is linked to SARS-like coronavirus spike protein, preferably an RBD thereof, or to a peptide comprising at least one epitope thereof, most preferably an epitope of SARS-CoV-2.

Preferred polypeptides or proteins including epitopes of such polypeptides or proteins, respectively, for use in the present invention are selected from SEQ ID NO: 1 to 14.

Preferred sequences for use in the prevention and/or treatment of influenza:

Sequences based on H1N1-G4-EA HA1

1) HA1 with C-terminal His-tag (in italic)
and secretion signal (secretion signal
underlined)
(SEQ ID NO: 1)
MNFGLRLIFLVLTLKGVQAMDTICVGYHANNSTDTVDTIL
EKNVTVTHSVNLLENSHNGKLCSLNGKIPLQLGNCNVAGW
ILGNPKCDLLLTANSWSYIIETSNSKNGACYPGEFADYEE
LKEQLSTVSSFERFEIFPKATSWPNHDTTRGTTVACSHSG
ANSFYRNLLWIVKKGNSYPKLSKSYTNNKGKEVLVIWGVH
HPPTDSDQQTLYQNNHTYVSVGSSKYYKRFTPEIVARPKV
REQAGRMNYYWTLLDQGDTITFEATGNLIAPWHAFALKKG
SSSGIMRSDAQVHNCTTKCQTPHGALKGNLPFQNVHPVTI
GKCPKYVKSTQLRMATGLRNIPSIQSRGENLYFQGGGSHH
HHHHHHHH
2) HA1 without His-tag and without secretion
signal (M at pos. 1 is optional)
(SEQ ID NO: 2)
(M)DTICVGYHANNSTDTVDTILEKNVTVTHSVNLLENSHN
GKLCSLNGKIPLQLGNCNVAGWILGNPKCDLLLTANSWSYI
IETSNSKNGACYPGEFADYEELKEQLSTVSSFERFEIFPKA
TSWPNHDTTRGTTVACSHSGANSFYRNLLWIVKKGNSYPK
LSKSYTNNKGKEVLVIWGVHHPPTDSDQQTLYQNNHTYVS
VGSSKYYKRFTPEIVARPKVREQAGRMNYYWTLLDQGDTI
TFEATGNLIAPWHAFALKKGSSSGIMRSDAQVHNCTTKCQ
TPHGALKGNLPFQNVHPVTIGKCPKYVKSTQLRMATGLRN
IPSIQSRG
3) HA1-C-CPE with N-terminal His-tag
(in italic) and secretion signal
(secretion signal underlined)
(SEQ ID NO: 3)
MNFGLRLIFLVLTLKGVQAMGHHHHHHHHHENLYFQGGGG
SGMDTICVGYHANNSTDTVDTILEKNVTVTHSVNLLENSH
NGKLCSLNGKIPLQLGNCNVAGWILGNPKCDLLLTANSWS
YIIETSNSKNGACYPGEFADYEELKEQLSTVSSFERFEIF
PKATSWPNHDTTRGTTVACSHSGANSFYRNLLWIVKKGNS
YPKLSKSYTNNKGKEVLVIWGVHHPPTDSDQQTLYQNNHT
YVSVGSSKYYKRFTPEIVARPKVREQAGRMNYYWTLLDQG
DTITFEATGNLIAPWHAFALKKGSSSGIMRSDAQVHNCTT
KCQTPHGALKGNLPFQNVHPVTIGKCPKYVKSTQLRMATG
LRNIPSIQSRGGGGSDIEKEILDLAAATERLNLTDALNSN
PAGNLYDWRSSNSYPWTQKLNLHLTITATGQKYRILASKI
VDFNIYSNNFNNLVKLEQSLGDGVKDHYVDISLDAGQYVL
VMKANSSYSGNYPYSILFQKF
4) HA1-C-CPE without His-tag and without
secretion signal (M at pos. 1 is
optional)
(SEQ ID NO: 4)
(M)DTICVGYHANNSTDTVDTILEKNVTVTHSVNLLENSHN
GKLCSLNGKIPLQLGNCNVAGWILGNPKCDLLLTANSWSYI
IETSNSKNGACYPGEFADYEELKEQLSTVSSFERFEIFPKA
TSWPNHDTTRGTTVACSHSGANSFYRNLLWIVKKGNSYPK
LSKSYTNNKGKEVLVIWGVHHPPTDSDQQTLYQNNHTYVS
VGSSKYYKRFTPEIVARPKVREQAGRMNYYWTLLDQGDTI
TFEATGNLIAPWHAFALKKGSSSGIMRSDAQVHNCTTKCQ
TPHGALKGNLPFQNVHPVTIGKCPKYVKSTQLRMATGLRN
IPSIQSRGGGGSDIEKEILDLAAATERLNLTDALNSNPAG
NLYDWRSSNSYPWTQKLNLHLTITATGQKYRILASKIVDF
NIYSNNFNNLVKLEQSLGDGVKDHYVDISLDAGQYVLVMK
ANSSYSGNYPYSILFQKF

Preferred sequences for use in the prevention and/or treatment of SARS-CoV-2 infections:

Sequences SARS-CoV-2 Spike S1 RBD

5) RBD with C-terminal His-tag (in italic)
and secretion signal (secretion signal
underlined)
(SEQ ID NO: 5)
MNFGLRLIFLVLTLKGVQAMRVQPTESIVRFPNITNLCPFGEVFNATRFASVYAWNR
KRISNCVADYSVLYNSASFSTFKCYGVSPTKLNDLCFTNVYADSFVIRGDEVRQIAP
GQTGKIADYNYKLPDDFTGCVIAWNSNNLDSKVGGNYNYLYRLFRKSNLKPFERDIS
TEIYQAGSTPCNGVEGFNCYFPLQSYGFQPTNGVGYQPYRVVVLSFELLHAPATVCG
PKKSTNLVKNKCVNFVLVPRGSAAALEHHHHHHHH
6) RBD without His-tag and without secretion
signal (M at pos. 1 is optional)
(SEQ ID NO: 6)
(M)RVQPTESIVRFPNITNLCPFGEVFNATRFASVYAWNRKRISNCVADYSVLYNSA
SFSTFKCYGVSPTKLNDLCFTNVYADSFVIRGDEVRQIAPGQTGKIADYNYKLPDDF
TGCVIAWNSNNLDSKVGGNYNYLYRLFRKSNLKPFERDISTEIYQAGSTPCNGVEGF
NCYFPLQSYGFQPTNGVGYQPYRVVVLSFELLHAPATVCGPKKSTNLVKNKCVNF
7) RBD-C-CPE with N-terminal His-tag (in
italic) and secretion signal
(secretion signal underlined)
(SEQ ID NO: 7)
MNFGLRLIFLVLTLKGVQAMGHHHHHHHHHENLYFQGGGGSGMRVQPTESIVRFPNI
TNLCPFGEVENATRFASVYAWNRKRISNCVADYSVLYNSASFSTFKCYGVSPTKLND
LCFTNVYADSFVIRGDEVRQIAPGQTGKIADYNYKLPDDFTGCVIAWNSNNLDSKVG
GNYNYLYRLFRKSNLKPFERDISTEIYQAGSTPCNGVEGFNCYFPLQSYGFQPTNGV
GYQPYRVVVLSFELLHAPATVCGPKKSTNLVKNKCVNFGGGGSDIEKEILDLAAATE
RLNLTDALNSNPAGNLYDWRSSNSYPWTQKLNLHLTITATGQKYRILASKIVDFNIY
SNNFNNLVKLEQSLGDGVKDHYVDISLDAGQYVLVMKANSSYSGNYPYSILFQKF
8) RBD-C-CPE without His-tag and without
secretion signal (M at pos. 1 is
optional)
(SEQ ID NO: 8)
(M)RVQPTESIVRFPNITNLCPFGEVFNATRFASVYAWNRKRISNCVADYSVLYNSA
SFSTFKCYGVSPTKLNDLCFTNVYADSFVIRGDEVRQIAPGQTGKIADYNYKLPDDF
TGCVIAWNSNNLDSKVGGNYNYLYRLFRKSNLKPFERDISTEIYQAGSTPCNGVEGF
NCYFPLQSYGFQPTNGVGYQPYRVVVLSFELLHAPATVCGPKKSTNLVKNKCVNFGG
GGSDIEKEILDLAAATERLNLTDALNSNPAGNLYDWRSSNSYPWTQKLNLHLTITAT
GQKYRILASKIVDFNIYSNNFNNLVKLEQSLGDGVKDHYVDISLDAGQYVLVMKANS
SYSGNYPYSILFQKF

Protein Sequences Full Length SARS-CoV-2 Spike (Stabilized Version)

9) Stabilized Spike trimer with C-terminal
His/Strep-tag including trimerization
domain (in italic) and secretion signal
(secretion signal underlined)
(SEQ ID NO: 9)
MALPVTALLLPLALLLHAARPMVNLTTRTQLPPAYTNSFTRGVYYPDKVFRSSVLHS
TQDLFLPFFSNVTWFHAIHVSGTNGTKRFDNPVLPFNDGVYFASTEKSNIIRGWIFG
TTLDSKTQSLLIVNNATNVVIKVCEFQFCNDPFLGVYYHKNNKSWMESEFRVYSSAN
NCTFEYVSQPFLMDLEGKQGNFKNLREFVFKNIDGYFKIYSKHTPINLVRDLPQGFS
ALEPLVDLPIGINITRFQTLLALHRSYLTPGDSSSGWTAGAAAYYVGYLQPRTFLLK
YNENGTITDAVDCALDPLSETKCTLKSFTVEKGIYQTSNFRVQPTESIVRFPNITNL
CPFGEVFNATRFASVYAWNRKRISNCVADYSVLYNSASFSTFKCYGVSPTKLNDLCF
TNVYADSFVIRGDEVRQIAPGQTGKIADYNYKLPDDFTGCVIAWNSNNLDSKVGGNY
NYLYRLFRKSNLKPFERDISTEIYQAGSTPCNGVEGFNCYFPLQSYGFQPTNGVGYQ
PYRVVVLSFELLHAPATVCGPKKSTNLVKNKCVNFNFNGLTGTGVLTESNKKFLPFQ
QFGRDIADTTDAVRDPQTLEILDITPCSFGGVSVITPGTNTSNQVAVLYQDVNCTEV
PVAIHADQLTPTWRVYSTGSNVFQTRAGCLIGAEHVNNSYECDIPIGAGICASYQTQ
TNSPGASVASQSIIAYTMSLGAENSVAYSNNSIAIPTNFTISVTTEILPVSMTKTSV
DCTMYICGDSTECSNLLLQYGSFCTQLNRALTGIAVEQDKNTQEVFAQVKQIYKTPP
IKDFGGFNFSQILPDPSKPSKRSFIEDLLFNKVTLADAGFIKQYGDCLGDIAARDLI
CAQKFNGLTVLPPLLTDEMIAQYTSALLAGTITSGWTFGAGAALQIPFAMQMAYRFN
GIGVTQNVLYENQKLIANQFNSAIGKIQDSLSSTASALGKLQDVVNQNAQALNTLVK
QLSSNFGAISSVLNDILSRLDPPEAEVQIDRLITGRLQSLQTYVTQQLIRAAEIRAS
ANLAATKMSECVLGQSKRVDFCGKGYHLMSFPQSAPHGVVFLHVTYVPAQEKNFTTA
PAICHDGKAHFPREGVFVSNGTHWFVTQRNFYEPQIITTDNTFVSGNCDVVIGIVNN
TVYDPLQPELDSFKEELDKYFKNHTSPDVDLGDISGINASVVNIQKEIDRLNEVAKN
LNESLIDLQELGKYEQYIKGSGYIPEAPRDGQAYVRKDGEWVLLSTFLGENLYFQGG
SAWSHPQFEKGGGSGGGSGGSAWSHPQFEKGSHHHHHHHH
10) Stabilized Spike trimer without His/
Strep-tag, including trimerization
domain (in italic) and without secretion
signal (M at pos. 1 is optional)
(SEQ ID NO: 10)
(M)VNLTTRTQLPPAYTNSFTRGVYYPDKVFRSSVLHSTQDLFLPFFSNVTWFHAIH
VSGTNGTKRFDNPVLPFNDGVYFASTEKSNIIRGWIFGTTLDSKTQSLLIVNNATNV
VIKVCEFQFCNDPFLGVYYHKNNKSWMESEFRVYSSANNCTFEYVSQPFLMDLEGKQ
GNFKNLREFVFKNIDGYFKIYSKHTPINLVRDLPQGFSALEPLVDLPIGINITRFQT
LLALHRSYLTPGDSSSGWTAGAAAYYVGYLQPRTFLLKYNENGTITDAVDCALDPLS
ETKCTLKSFTVEKGIYQTSNFRVQPTESIVRFPNITNLCPFGEVFNATRFASVYAWN
RKRISNCVADYSVLYNSASFSTFKCYGVSPTKLNDLCFTNVYADSFVIRGDEVRQIA
PGQTGKIADYNYKLPDDFTGCVIAWNSNNLDSKVGGNYNYLYRLFRKSNLKPFERDI
STEIYQAGSTPCNGVEGFNCYFPLQSYGFQPTNGVGYQPYRVVVLSFELLHAPATVC
GPKKSTNLVKNKCVNFNFNGLTGTGVLTESNKKFLPFQQFGRDIADTTDAVRDPQTL
EILDITPCSFGGVSVITPGTNTSNQVAVLYQDVNCTEVPVAIHADQLTPTWRVYSTG
SNVFQTRAGCLIGAEHVNNSYECDIPIGAGICASYQTQTNSPGASVASQSIIAYTMS
LGAENSVAYSNNSIAIPTNFTISVTTEILPVSMTKTSVDCTMYICGDSTECSNLLLQ
YGSFCTQLNRALTGIAVEQDKNTQEVFAQVKQTYKTPPIKDFGGFNFSQILPDPSKP
SKRSFIEDLLFNKVTLADAGFIKQYGDCLGDIAARDLICAQKFNGLTVLPPLLTDEM
IAQYTSALLAGTITSGWTFGAGAALQIPFAMQMAYRENGIGVTQNVLYENQKLIANQ
FNSAIGKIQDSLSSTASALGKLQDVVNQNAQALNTLVKQLSSNFGAISSVLNDILSR
LDPPEAEVQIDRLITGRLQSLQTYVTQQLIRAAEIRASANLAATKMSECVLGQSKRV
DFCGKGYHLMSFPQSAPHGVVFLHVTYVPAQEKNFTTAPAICHDGKAHFPREGVFVS
NGTHWFVTQRNFYEPQIITTDNTFVSGNCDVVIGIVNNTVYDPLQPELDSFKEELDK
YFKNHTSPDVDLGDISGINASVVNIQKEIDRLNEVAKNLNESLIDLQELGKYEQYIK
GSGYIPEAPRDGQAYVRKDGEWVLLSTFL
11) Stabilized Spike-C-CPE trimer with
N-terminal His-tag (in italic), C-
terminal trimerization domain and
secretion signal (secretion signal
underlined)
(SEQ ID NO: 11)
MNFGLRLIFLVLTLKGVQAMGHHHHHHHHHENLYFQGGGGSGMVNLTTRTQLPPAYT
NSFTRGVYYPDKVFRSSVLHSTQDLFLPFFSNVTWFHAIHVSGTNGTKRFDNPVLPF
NDGVYFASTEKSNIIRGWIFGTTLDSKTQSLLIVNNATNVVIKVCEFQFCNDPFLGV
YYHKNNKSWMESEFRVYSSANNCTFEYVSQPFLMDLEGKQGNFKNLREFVFKNIDGY
FKIYSKHTPINLVRDLPQGFSALEPLVDLPIGINITRFQTLLALHRSYLTPGDSSSG
WTAGAAAYYVGYLQPRTFLLKYNENGTITDAVDCALDPLSETKCTLKSFTVEKGIYQ
TSNFRVQPTESIVRFPNITNLCPFGEVFNATRFASVYAWNRKRISNCVADYSVLYNS
ASFSTFKCYGVSPTKLNDLCFTNVYADSFVIRGDEVRQIAPGQTGKIADYNYKLPDD
FTGCVIAWNSNNLDSKVGGNYNYLYRLFRKSNLKPFERDISTEIYQAGSTPCNGVEG
FNCYFPLQSYGFQPTNGVGYQPYRVVVLSFELLHAPATVCGPKKSTNLVKNKCVNFN
FNGLTGTGVLTESNKKFLPFQQFGRDIADTTDAVRDPQTLEILDITPCSFGGVSVIT
PGTNTSNQVAVLYQDVNCTEVPVAIHADQLTPTWRVYSTGSNVFQTRAGCLIGAEHV
NNSYECDIPIGAGICASYQTQTNSPGASVASQSIIAYTMSLGAENSVAYSNNSIAIP
TNFTISVTTEILPVSMTKTSVDCTMYICGDSTECSNLLLQYGSFCTQLNRALTGIAV
DAGFIKQYGDCLGDIAARDLICAQKFNGLTVLPPLLTDEMIAQYTSALLAGTITSGW
TFGAGAALQIPFAMQMAYRFNGIGVTQNVLYENQKLIANQFNSAIGKIQDSLSSTAS
ALGKLQDVVNQNAQALNTLVKQLSSNFGAISSVLNDILSRLDPPEAEVQIDRLITGR
LQSLQTYVTQQLIRAAEIRASANLAATKMSECVLGQSKRVDFCGKGYHLMSFPQSAP
HGVVFLHVTYVPAQEKNFTTAPAICHDGKAHFPREGVFVSNGTHWFVTQRNFYEPQI
ITTDNTFVSGNCDVVIGIVNNTVYDPLQPELDSFKEELDKYFKNHTSPDVDLGDISG
INASVVNIQKEIDRLNEVAKNLNESLIDLQELGKYEQYIKGSGYIPEAPRDGQAYVR
KDGEWVLLSTFLGGGSDIEKEILDLAAATERLNLTDALNSNPAGNLYDWRSSNSYPW
TQKLNLHLTITATGQKYRILASKIVDFNIYSNNFNNLVKLEQSLGDGVKDHYVDISL
DAGQYVLVMKANSSYSGNYPYSILFQKF
12) Stabilized Spike-C-CPE trimer without
His-tag domain and without
secretion signal (M at pos 1 is optional),
trimerization domain (in italic)
retained
(SEQ ID NO: 12)
(M)VNLTTRTQLPPAYTNSFTRGVYYPDKVFRSSVLHSTQDLFLPFFSNVTWFHAIH
VSGTNGTKRFDNPVLPFNDGVYFASTEKSNIIRGWIFGTTLDSKTQSLLIVNNATNV
VIKVCEFQFCNDPFLGVYYHKNNKSWMESEFRVYSSANNCTFEYVSQPFLMDLEGKQ
GNFKNLREFVFKNIDGYFKIYSKHTPINLVRDLPQGFSALEPLVDLPIGINITRFQT
LLALHRSYLTPGDSSSGWTAGAAAYYVGYLQPRTFLLKYNENGTITDAVDCALDPLS
ETKCTLKSFTVEKGIYQTSNFRVQPTESIVRFPNITNLCPFGEVFNATRFASVYAWN
RKRISNCVADYSVLYNSASFSTFKCYGVSPTKLNDLCFTNVYADSFVIRGDEVRQIA
PGQTGKIADYNYKLPDDFTGCVIAWNSNNLDSKVGGNYNYLYRLFRKSNLKPFERDI
STEIYQAGSTPCNGVEGFNCYFPLQSYGFQPTNGVGYQPYRVVVLSFELLHAPATVC
GPKKSTNLVKNKCVNFNFNGLTGTGVLTESNKKFLPFQQFGRDIADTTDAVRDPQTL
EILDITPCSFGGVSVITPGTNTSNQVAVLYQDVNCTEVPVAIHADQLTPTWRVYSTG
SNVFQTRAGCLIGAEHVNNSYECDIPIGAGICASYQTQTNSPGASVASQSIIAYTMS
LGAENSVAYSNNSIAIPTNFTISVTTEILPVSMTKTSVDCTMYICGDSTECSNLLLQ
YGSFCTQLNRALTGIAVEQDKNTQEVFAQVKQIYKTPPIKDFGGFNFSQILPDPSKP
SKRSFIEDLLFNKVTLADAGFIKQYGDCLGDIAARDLICAQKFNGLTVLPPLLTDEM
IAQYTSALLAGTITSGWTFGAGAALQIPFAMQMAYRENGIGVTQNVLYENQKLIANQ
FNSAIGKIQDSLSSTASALGKLQDVVNQNAQALNTLVKQLSSNFGAISSVLNDILSR
LDPPEAEVQIDRLITGRLQSLQTYVTQQLIRAAEIRASANLAATKMSECVLGQSKRV
DFCGKGYHLMSFPQSAPHGVVFLHVTYVPAQEKNFTTAPAICHDGKAHFPREGVFVS
NGTHWFVTQRNFYEPQIITTDNTFVSGNCDVVIGIVNNTVYDPLQPELDSFKEELDK
YFKNHTSPDVDLGDISGINASVVNIQKEIDRLNEVAKNLNESLIDLQELGKYEQYIK
GSGYIPEAPRDGQAYVRKDGEWVLLSTFLGGGSDIEKEILDLAAATERLNLTDALNS
NPAGNLYDWRSSNSYPWTQKLNLHLTITATGQKYRILASKIVDFNIYSNNFNNLVKL
EQSLGDGVKDHYVDISLDAGQYVLVMKANSSYSGNYPYSILFQKF
13) Stabilized Spike-C-CPE monomer with
N-terminal His-tag (in italic) and
secretion signal (secretion signal
underlined)
(SEQ ID NO: 13)
MNFGLRLIFLVLTLKGVQAMGHHHHHHHHHENLYFQGGGGSGMVNLTTRTQLPPAYT
NSFTRGVYYPDKVFRSSVLHSTQDLFLPFFSNVTWFHAIHVSGTNGTKRFDNPVLPF
NDGVYFASTEKSNIIRGWIFGTTLDSKTQSLLIVNNATNVVIKVCEFQFCNDPFLGV
YYHKNNKSWMESEFRVYSSANNCTFEYVSQPFLMDLEGKQGNFKNLREFVFKNIDGY
FKIYSKHTPINLVRDLPQGFSALEPLVDLPIGINITRFQTLLALHRSYLTPGDSSSG
WTAGAAAYYVGYLQPRTFLLKYNENGTITDAVDCALDPLSETKCTLKSFTVEKGIYQ
TSNFRVQPTESIVRFPNITNLCPFGEVFNATRFASVYAWNRKRISNCVADYSVLYNS
ASFSTFKCYGVSPTKLNDLCFTNVYADSFVIRGDEVRQIAPGQTGKIADYNYKLPDD
FTGCVIAWNSNNLDSKVGGNYNYLYRLFRKSNLKPFERDISTEIYQAGSTPCNGVEG
FNCYFPLQSYGFQPTNGVGYQPYRVVVLSFELLHAPATVCGPKKSTNLVKNKCVNFN
FNGLTGTGVLTESNKKFLPFQQFGRDIADTTDAVRDPQTLEILDITPCSFGGVSVIT
PGTNTSNQVAVLYQDVNCTEVPVAIHADQLTPTWRVYSTGSNVFQTRAGCLIGAEHV
NNSYECDIPIGAGICASYQTQTNSPGASVASQSIIAYTMSLGAENSVAYSNNSIAIP
TNFTISVTTEILPVSMTKTSVDCTMYICGDSTECSNLLLQYGSFCTQLNRALTGIAV
DAGFIKQYGDCLGDIAARDLICAQKFNGLTVLPPLLTDEMIAQYTSALLAGTITSGW
TFGAGAALQIPFAMQMAYRENGIGVTQNVLYENQKLIANQFNSAIGKIQDSLSSTAS
ALGKLQDVVNQNAQALNTLVKQLSSNFGAISSVLNDILSRLDPPEAEVQIDRLITGR
LQSLQTYVTQQLIRAAEIRASANLAATKMSECVLGQSKRVDFCGKGYHLMSFPQSAP
HGVVFLHVTYVPAQEKNFTTAPAICHDGKAHFPREGVFVSNGTHWFVTQRNFYEPQI
ITTDNTFVSGNCDVVIGIVNNTVYDPLQPELDSFKEELDKYFKNHTSPDVDLGDISG
INASVVNIQKEIDRLNEVAKNLNESLIDLQELGKYEQYIKGGGSDIEKEILDLAAAT
ERLNLTDALNSNPAGNLYDWRSSNSYPWTQKLNLHLTITATGQKYRILASKIVDFNI
YSNNFNNLVKLEQSLGDGVKDHYVDISLDAGQYVLVMKANSSYSGNYPYSILFQKF
14) Stabilized Spike-C-CPE monomer
without His-tag and without secretion
signal (M at pos. 1 is optional)
(SEQ ID NO: 14)
(M)VNLTTRTQLPPAYTNSFTRGVYYPDKVFRSSVLHSTQDLFLPFFSNVTWFHAIH
VSGTNGTKRFDNPVLPFNDGVYFASTEKSNIIRGWIFGTTLDSKTQSLLIVNNATNV
VIKVCEFQFCNDPFLGVYYHKNNKSWMESEFRVYSSANNCTFEYVSQPFLMDLEGKQ
GNFKNLREFVFKNIDGYFKIYSKHTPINLVRDLPQGFSALEPLVDLPIGINITRFQT
LLALHRSYLTPGDSSSGWTAGAAAYYVGYLQPRTFLLKYNENGTITDAVDCALDPLS
ETKCTLKSFTVEKGIYQTSNFRVQPTESIVRFPNITNLCPFGEVFNATRFASVYAWN
RKRISNCVADYSVLYNSASFSTFKCYGVSPTKLNDLCFTNVYADSFVIRGDEVRQIA
PGQTGKIADYNYKLPDDFTGCVIAWNSNNLDSKVGGNYNYLYRLFRKSNLKPFERDI
STEIYQAGSTPCNGVEGFNCYFPLQSYGFQPTNGVGYQPYRVVVLSFELLHAPATVC
GPKKSTNLVKNKCVNFNFNGLTGTGVLTESNKKFLPFQQFGRDIADTTDAVRDPQTL
EILDITPCSFGGVSVITPGTNTSNQVAVLYQDVNCTEVPVAIHADQLTPTWRVYSTG
SNVFQTRAGCLIGAEHVNNSYECDIPIGAGICASYQTQTNSPGASVASQSIIAYTMS
LGAENSVAYSNNSIAIPTNFTISVTTEILPVSMTKTSVDCTMYICGDSTECSNLLLQ
YGSFCTQLNRALTGIAVEQDKNTQEVFAQVKQIYKTPPIKDFGGFNFSQILPDPSKP
SKRSFIEDLLFNKVTLADAGFIKQYGDCLGDIAARDLICAQKFNGLTVLPPLLTDEM
IAQYTSALLAGTITSGWTFGAGAALQIPFAMQMAYRFNGIGVTQNVLYENQKLIANQ
FNSAIGKIQDSLSSTASALGKLQDVVNQNAQALNTLVKQLSSNFGAISSVLNDILSR
LDPPEAEVQIDRLITGRLQSLQTYVTQQLIRAAEIRASANLAATKMSECVLGQSKRV
DFCGKGYHLMSFPQSAPHGVVFLHVTYVPAQEKNFTTAPAICHDGKAHFPREGVFVS
NGTHWFVTQRNFYEPQIITTDNTFVSGNCDVVIGIVNNTVYDPLQPELDSFKEELDK
YFKNHTSPDVDLGDISGINASVVNIQKEIDRLNEVAKNLNESLIDLQELGKYEQYIK
GGGSDIEKEILDLAAATERLNLTDALNSNPAGNLYDWRSSNSYPWTQKLNLHLTITA
TGQKYRILASKIVDFNIYSNNFNNLVKLEQSLGDGVKDHYVDISLDAGQYVLVMKAN
SSYSGNYPYSILFQKF

Nucleic acids encoding antigenic peptide components for use in the invention are preferably mRNAs. Preferably, mRNAs for use in the invention are codon optimized. It is also preferred that the mRNA contains one or more nucleotide analogues.

The chemical modification of the nucleotide analogue in comparison to the natural occurring nucleotide may be at the ribose, phosphate and/or base moiety. With respect to molecules having an increased stability, especially with respect to RNA degrading enzymes, modifications at the backbone, i. e. the ribose and/or phosphate moieties, are especially preferred.

Preferred examples of ribose-modified ribonucleotides are analogues wherein the 2′—OH group is replaced by a group selected from H, OR, R, halo, SH, SR, NH2, NHR, NR2, or CN with R being CrC6 alkyl, alkenyl or alkynyl and halo being F, Cl, Br or I. It is clear for the person skilled in the art that the term “modified ribonucleotide” also includes 2′-deoxy derivatives, such as 2′-0-methyl derivatives, which may at several instances also be termed “deoxynucleotides”.

As mentioned before, the at least one modified ribonucleotide may be selected from analogues having a chemical modification at the base moiety. Examples of such analogues include, but are not limited to, 5-aminoallyl-uridine, 6-aza-uridine, 8-aza-adenosine, 5-bromo-uridine, 7-deaza-adenine, 7-deaza-guanine, N6-methyl-adenine, 5-methyl-cytidine, pseudo-uridine, and 4-thio-uridine. Pseudo-uridine is a particularly preferred nucleotide analogue in the context of the present invention. In preferred embodiments of the invention substantially all of the uridine nucleotides present in a corresponding natural coding RNA, in particular mRNA, are replaced by pseudo-uridine nucleotides.

Examples of backbone-modified ribonucleotides wherein the phosphoester group between adjacent ribonucleotides is modified are phosphothioate groups stabilized by inclusion of nucleotide analogues.

mRNA-containing compositions of the inventions or such compositions for use in the invention preferably contain the mRNA formulated together with an mRNA delivery vehicle such as a lipid-based delivery vehicle, a polymer-based delivery vehicle or a hybrid lipid/polymer vehicle such as those disclosed in Table 1 id Xu et al. (2020). In highly preferred embodiments of the invention such compositions comprise the mRNA formulated as a lipid nanoparticle (LNP).

Preferred mRNA Delivery Vehicles (or mRNA Delivery Systems)

The second essential component of pharmaceutical compositions as defined herein are adjuvants eliciting an innate immune response in the human subject, in particular for avoiding a protection gap as outlined above, preferably in case of SARS-like coronavirus infection, most preferably SARS-CoV-2 infections, or influenza infections. Adjuvants for use in the invention are typically selected from pathogen-associated molecular patterns (PAMPs), in particular ligands of pattern recognition receptors (PRRs), preferably selected from Toll-like receptor (TLR) agonists, RIG-I agonists and/or STING agonists.

In one preferred embodiment of the invention, one of the adjuvants or the adjuvant, respectively, is an agonist of TLR-3 and/or RIG-I. In other embodiments, one of the adjuvant or the adjuvant, respectively, is selected from TLR-4, TLR-7, TLR-8, TLR-9 and STING agonists.

Preferred TLR-3 agonists of the invention are double-stranded RNAs (dsRNAs) of at least about 45 bp. Preferably, the dsRNA has a length of about 45 to about 200 bp, more preferably from about 45 to about 100 bp. Preferably, the dsRNA for use as an adjuvant in the invention is a dsRNA having two blunt ends, preferably a perfectly annealed dsRNA having two blunt ends, or is a perfectly annealed dsRNA having one blunt end and having a single-stranded overhang of 1 to 5, preferably 1 to 3 nt, at the other end of the dsRNA. For additionally triggering RIG-I (i.e. being a TLR-3 agonist and a RIG-I-agonist), the dsRNA comprises a free 5′-triphophate group at least one double-stranded end. Preferred dsRNAs for use as adjuvant in the present invention are disclosed in WO 2013/064584 A1 (in particular, dsRNAs according to any one of claims 1 to 14 thereof) and WO 2015/091578 A1 (in particular, dsRNAs and compositions thereof according to any one of claims 1 to 9 thereof), the disclosure contents of which are hereby included by reference.

In preferred embodiments of the invention the dsRNA, more preferably a dsRNA as disclosed in WO 2013/064584 A1 contains one or more nucleotide analogues as already outlined above for coding RNAs for use in the invention.

The pharmaceutical composition can include further adjuvants known in the art of vaccination compositions.

The composition for use in the invention preferably comprises at least one pharmaceutically acceptable carrier. Preferred carriers for use in the invention include, but are not limited to, chitosan, chitosan derivatives, polyethyleneimine, PGLA, cationic liposomes, saccharides such as sucrose, trehalose and mannitol, sodium succinate, amino acids such as arginine and histidine and mixtures of two or more thereof.

Other carriers for use in the invention are selected from chitosan, derivatives and salts thereof. Preferred chitosan derivatives for use in the invention include, but are not limited to, crosslinked chitosan, annilin-chitosan-copolymers, PEG-chitosan-copolymers, N- and/or O-carboxymethylchitosan, hydroxypropylchitosan, N- and/or O-acylchitosan, N-alkylchitosans and mixtures thereof.

Particularly preferred carriers for use in the invention are selected from saccharides, preferably trehalose, mannitol and sucrose and mixtures of two or more thereof. The inventors have determined that saccharides such as trehalose, mannitol and sucrose have a stabilizing effect on antigenic components for use in the present invention, in particular antigenic proteins or protein fragments as described herein, especially preferred the Spike protein of SARS-CoV-2, more preferably the RBD fragment thereof. The stabilizing effect of such saccharides is particularly pronounced for the lyophilized forms of antigenic structures as disclosed herein, in particular the Spike protein of SARS-CoV-2, and especially the RBD fragment thereof. Most preferred, the saccharide is trehalose.

Inventive composition comprising an antigenic component as defined herein, preferably a fusion of an antigenic component linked to a mucosa-targeting moiety, more preferably C-CPE, and a dsRNA TLR-3 agonist, more particular a dsRNA TLR-3 agonist of the preferred embodiments as described herein are also denoted as “XPOVAX” compositions or vaccines, respectively.

The pharmaceutical composition preferably comprises a mixture of two or more carriers, preferably selected from those as exemplified above, more preferably a chitosan or derivative or salt thereof (such as one or more preferred embodiments as stated above) and a further carrier, such as preferably trehalose, sucrose, arginine, mannitol, sodium succinate or histidine or a mixture of two or more thereof. As regards mannitol and arginine (in particular L-arginine), it is preferred to use a mixture of mannitol and arginine, preferably L-arginine, which is particularly useful in compositions for intra-nasal application.

The pharmaceutical composition may be prepared in various forms as long as the specific form complies with the delivery of the composition in vaccine applications. For example, the pharmaceutical composition may be in liquid form comprising a solution or suspension of the components in a suitable liquid such as water, preferably sterilized water as typically used for injection, aqueous saline, Ringer, Ringer Lactate and other known formulation aids for liquids. In other embodiments, the composition can be in solid of half-solid form such as a powder, preferably a freeze-dried powder, small granules, micro- or nanoparticles. For intra-nasal applications, liquid applications forms are used in intranasal delivery devices such as a nebulizer comprising a container, for example a glass or, more preferred, plastic vial, containing the liquid composition, and a nebulizer or spray element typically comprising a pump mechanism so as to prepare and eject a fine aerosol of the composition into the nose of the subject. In other embodiments of the invention, the pharmaceutical composition can be administered, preferably for intra-nasal application, in solid form, preferably as a powder, preferably a lyophilized powder (or other finely grained, milled or divided particulate form).

For application, especially by intra-nasal application, a pharmaceutical composition in solid form such a powder, more preferably, a lyophilized powder or other small particulate form, may be reconstituted by addition of a desired liquid such as those as exemplified above, in order to provide the final composition for administration. For such applications, which are also useful for treatments as defined herein by self-administration, the composition in solid form, e.g. a lyophilized powder, is present in a suitable container such as a glass or plastic vial, and the user is provided with the suitable liquid for reconstitution in a second container. The user, e.g. the subject in the case of self-administration, can combine the contents of the containers, preferably by adding the liquid to the solid composition so as to provide the final composition, for example as a solution or suspension, for administration.

The present invention is also directed to certain pharmaceutical compositions as defined herein as such, namely pharmaceutical compositions as defined herein before comprising (a) at least one antigenic component of a virus causing a respiratory infection, said at least one antigenic component being coupled to a mucosa-targeting moiety and/or a nucleic acid encoding at least one antigenic peptide component of a virus causing a respiratory infection linked to a mucosa-targeting moiety, and (b) one or more TLR-3 and/or RIG-I agonists.

The invention is also directed to an intra-nasal pharmaceutical composition comprising a nucleic acid encoding at least one antigenic peptide component of a virus causing a respiratory infection linked to a mucosa-targeting moiety. Preferably, the intra-nasal composition comprises an mRNA encoding a SARS-CoV-2 RBD linked to C-CPE.

Preferred embodiments of the components according to (a) and (b) have been defined and described herein-above. The pharmaceutical composition of the invention or for use in the invention is preferably in liquid or lyophilized form.

The present invention further relates to a pharmaceutical kit comprising an intranasal delivery device, e.g. a device for intra-nasal administration as exemplified above, and a unit dose of the pharmaceutical composition according to the invention.

A unit dose of a pharmaceutical composition according to the invention or for use in the invention typically comprises effective amounts of the antigenic component and of the one or more adjuvants such that the adjuvant elicits an innate immune response in the human subject and the antigenic viral component is present in an amount sufficient to generate antibodies against the virus.

As regards antigenic proteinaceous or peptide, respectively, components, a unit dose typically comprises about 10 μg to about 2000 μg, preferably about 10 μg to about 100 μg, more preferably about 10 μg to about 50 μg of said protein or peptide (such as oligopeptide or polypeptide), respectively. With respect to nucleic acid adjuvants for use in the invention, the pharmaceutical composition comprises a nucleic acid, such as the preferred TLR-3 agonists as outlined above, typically in an amount in the range of from about 0.1 μg per kg body weight to about 90 μg per kg body weight. In preferred embodiments of the invention, a unit dose of the pharmaceutical composition comprises about 10 μg to about 2000 μg, preferably from about 20 μg to about 1500 μg, more preferably about 50 to about 1000 μg of the nucleic acid adjuvant, preferably a dsRNA as described herein-above.

The present invention also provides a method for treatment and/or prevention of a human subject against viral respiratory infections comprising the step of administering an effective amount of a pharmaceutical composition as described herein to the human subject, wherein the composition is administered pre-exposure or post-exposure of said subject to a virus causing said viral respiratory infection, or to the subject which is at least suspected to have been exposed to a virus causing said viral respiratory infection, respectively. Preferred embodiments of administration routes, schedules and regimens, antigenic components, adjuvants, viruses, carriers, targeting moieties etc. have already been elaborated above.

The present invention further provides a method for producing a pharmaceutical composition as described herein comprising the step of combining an antigenic component of a virus as described herein and one or more adjuvants as described herein. The invention further relates to a pharmaceutical device comprising a filling an intranasal delivery device with a dose of the from the formulation, said dose being a suitable volume for intranasal administration.

The present invention also provides a dsRNA TLR-3 agonist, more preferred a dsRNA as disclosed in WO 2013/064584 A1, linked to a mucosa-targeting moiety, preferably as described herein, more preferred C-CPE. The invention also relates to composition comprising such constructs, in particular for immunization against viral infections. The present invention relates to such dsRNA/mucosa targeting moiety comprising pharmaceutical compositions, in particular vaccine compositions, for preventing or at least reducing the severity of, respectively, viral respiratory infections through application of said composition to a human subject post-exposure or at least presumed post-exposure of said subject to a virus causing said viral respiratory infections or pre-exposure of said subject to said virus. Preferred viruses, administration routes, further components of such pharmaceutical compositions comprising dsRNA/mucosa-targeting moiety constructs have already been elaborated herein above.

### BRIEF DESCRIPTION OF THE DRAWINGS

FIG. 1 shows a schematic diagram illustrating a vaccination scheme according to the prior art leaving a protection gap in humans against respiratory viral infections such as infections by SARS-CoV.

FIG. 2 shows an illustration of the vaccination scheme according to Example 2.

FIG. 3 shows an illustration of the experimental schedule of Example 3.

FIG. 4 is a graphic representation of results of Example 3 showing the Matrix effect of naïve mouse sera (n=5) in a Luminex assay.

FIGS. 5a to 5f are graphic representations of results of endpoint titrations of SARS-CoV-2 spike binding IgG antibodies.

FIGS. 6a to 6f are graphic representations of results of endpoint titrations of SARS-CoV-2 spike binding IgA antibodies.

FIGS. 7a to 7d are graphic representations of the results of wildtype SARS-CoV-2 pseudovirus (Wuhan-Hu-1 strain) neutralization with XPOVAX-SARS-CoV-2 immunized mouse sera.

FIG. 8 is a graphic representation of wildtype SARS-CoV2 pseudovirus neutralization with XPOVAX-SARS-CoV-2 immunized mouse bronchioalveolar lavage (BAL) samples of mice from group #7 of Example 4.

FIGS. 9a to 9c are graphic representations of SARS-CoV-2 Variants of Concern (UK and SA VOC) pseudovirus neutralization titers (% neutralization) with XPOVAX-SARS-CoV-2 immunized mouse sera or BAL from group #7 of Example 4.

FIG. 9d is a graphic representation of SARS-CoV-2 Variants of Concern (UK VoC, SA VoC) pseudovirus neutralization titers (IC50 values titres) with XPOVAX-SARS-CoV-2 immunized mouse sera from group #7 of Example 4.

FIG. 10 is a graphic representation of T-cell responses (IFNγ ELISPOT with splenocytes, bulk all T-cells & CD4, CD8 T-cells separated) in mice immunized with XPOVAX SARS-CoV-2 vaccine.

FIG. 11 shows an illustration of the experimental schedule of Example 4.

FIGS. 12a to 12e show graphic representations of the reactivity of XPOVAX Influenza/SARS combination vaccine immunized mouse sera (IgG) with recombinant SARS-CoV-2 spike protein (wildtype and VoC variants) in the Luminex assay. Individual sera of group #5 mice (n=5). S-Trimer alpha=UK VoC, S-Trimer beta=SA VoC.

FIGS. 13a to 13e show graphic representations of the reactivity of XPOVAX Influenza/SARS combination vaccine immunized mouse sera (IgG) with SARS-CoV-2 recombinant receptor binding domain (RBD, wildtype, and RBD variants) in the Luminex assay. Individual sera of group #5 mice (n=5).

FIGS. 14a to 14e show graphic representations of the reactivity of XPOVAX Influenza/SARS combination vaccine immunized mouse sera (IgG) with recombinant influenza virus H1-HA1 in the Luminex assay. Individual sera of group #5 mice (n=5).

FIGS. 15a to 15e show graphic representations of the reactivity of XPOVAX Influenza/SARS combination vaccine immunized mouse nasal lavage (IgG) with recombinant SARS-RBD and H1-HA1 in the Luminex assay. Individual sera of group #5 mice (n=5).

FIGS. 16a to 16e show graphic representations of the reactivity of XPOVAX SARS/CoV-2 combination vaccine immunized mouse bronchio-alveolar lavage (IgG) with recombinant SARS-RBD and H1-HA1 in the Luminex assay. Individual sera of group #5 mice (n=5).

FIGS. 17a to 17e show graphic representations of the reactivity of XPOVAX Influenza/SARS combination vaccine immunized mouse bronchio-alveolar lavage (IgA) with recombinant SARS-RBD and H1-HA1 in the Luminex assay. Individual sera of group #5 mice (n=5).

FIGS. 18a to 18c show graphic representations of the results of pseudovirus (PV) neutralization assays (pseudotyped with spike protein of SARS-SoV-2 strain Wuhan-Hu-1) with sera and BAL from XPOVXAX Influenza/SARS immunized mice.

FIGS. 19a and 19b show graphic representations of T-cell responses (IFNγ ELISPOT, bulk all T-cells, and CD4, CD8 T-cells separated) in mice immunized with XPOVAX Influenza or XPOVAX Influenza-SARS combination vaccine, as single or prime-boost application.

FIG. 20 shows a table of results of pseudovirus neutralization titers (IC₅₀) of XPOVAX-SARS-CoV-2 immunized mouse sera and BAL.

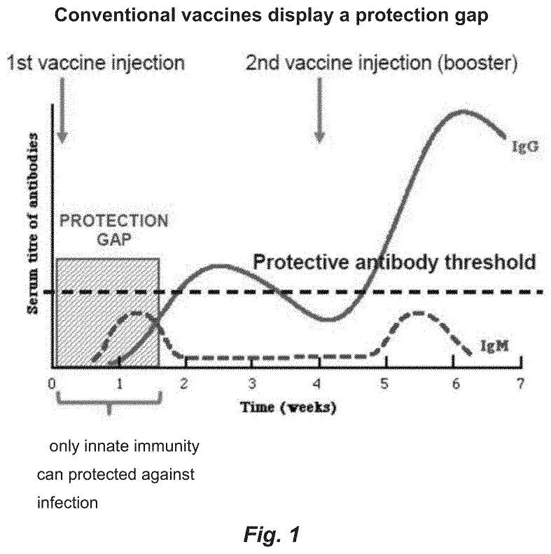

### DETAILED DESCRIPTION OF THE INVENTION

The following non-limiting examples further illustrate the present invention.

### EXAMPLES

### Example 1: Components for Pharmaceutical Composition

Component 1: Antigenic Component

The following constructs were provided by trenzyme GmbH, Konstanz, Germany.

Component 1.1: RBD of SARS-CoV-2

A his-tagged RBD of SARS-CoV-2 was prepared by recombinant expression in HEK293 cells. The construct was purified by one-step Ni-column chromatography. High-affinity binding was confirmed by SPR analysis (binding to human ACE2) (Kd of about 1 nM).

Component 1.2: Fusion protein construct of SARS-CoV-2 with C-CPE A fusion construct of his-tagged RBD of SARS-CoV with C-CPE was prepared by recombinant expression in HEK293 cells and purified as described for component 1.1.

Component 2: Adjuvant

A double-stranded TLR-3 agonist according to WO 2015/091578 A1 (dsRNA 100 bp, wherein one strand is polyC and the complementary strand is poly(G:I) and comprising a 5′-triphosphate at the polyC strand) was provided by RiboxX GmbH, Radebeul, Germany), hereinafter also denoted as “RIBOXXIM”.

Components 1 and 2 were combined and solubilized in water for injection (WFI) followed by lyophilization. The freeze-dried composition was reconstituted by addition of WFI.

The following compositions were prepared:

TABLE 1

| | Amount of | Amount of | Volume of liquid |
|---|---|---|---|
| | component | component | for reconstitution |
| Formulation | 1.1 [μg] | 2 [μg] | [μl] |
| 1 | 100 | 1000 | 100 |
| 2 | 100 | 500 | 100 |
| 3 | 100 | 250 | 100 |
| 4 | 100 | 125 | 100 |
| 5 | 100 | 61 | 100 |

### Example 2 Testing the Immune Response Elicited by Vaccination of Mice with RIBOXXIM and RBD-Fragment of SARS-CoV2 Spike Protein (Designated RBD) as Well as Modified RBD-Fragment (Designated RBD-PF), Both Provided by Trenzyme GmbH, Konstanz, Germany

Immunization of C57/BL6 mice with a combination of a TLR3 agonist (RIBOXXIM) and a viral structural protein of SARS-CoV-2 generates a local (IFN Type I, IgA) and systemic immune response (IgM, IgG)

Material and Methods

Reagents

Vaccine compositions containing component 2 of Example 1 (RIBOXXIM) and/or RBD-protein (component 1.1 of Example 1) or RBD-PF-Protein (component 1.2 of Example 1) and/or Chitosan are supplied in a volume of 250 μl as listed in the following Table 2:

TABLE 2

| Group | RIBOXXIM | Chitosan | RBD | RBD-PF | Total volume |
|---|---|---|---|---|---|
| # | (μg) | (μg) | (μg) | (μg) | (μl) |
| 1 | 50 | — | 10 | — | 300 |
| 2 | 50 | — | 10 | — | 300 |
| 3 | 50 | 10 | 10 | — | 300 |
| 4 | — | — | 10 | — | 300 |
| 5 | 50 | — | — | — | 300 |
| 6 | 50 | 10 | — | — | 300 |
| 7 | 50 | — | — | 10 | 300 |
| 8 | — | — | — | 10 | 300 |
| 9 (PBS) | — | — | — | — | 300 |

Collection of Samples

For collection of nasal swabs, nasosorption (Mucosal Diagnostics, Midhurst, UK) is used according to the instructions of the manufacturer.

Collection of blood samples is performed in the tail vein. Bronchoalveolar lavage and collection of splenocytes are performed after sacrifice of the animals.

Animals

C57/BL6 mice (n=5 per Group) are used.

Application of the Vaccine and Schedule

Each animal from Groups 1, 3, 4, 5, 6, 7, 8 and 9 receives 25 μl of the vaccine or the Vehicle (PBS, Group #8) in each nostril, corresponding to a total application of 50 μl per animal.

Animals from Group 2 receive 25 μl of the vaccine sub-cutaneous at day 0 and 25 μl of the vaccine intra-nasal at day 21.

Application occurs at day 0 and day 21.

For Groups 1, 3, 4, 5, 6, 7 and 9: nasal swabs are collected at days 0, 1, 3 and 5 and frozen at −20° C.

For Groups 1 to 9: blood is drawn at days 0 and day 21 and frozen at −20° C.

For Groups 1 to 9: at day 28, blood, BAL, nasal wash is collected and frozen at −20° C.

For Groups 1, 2, 3, 4, 7, 8, 9: at day 28, splenocytes are collected and frozen at −20° C.

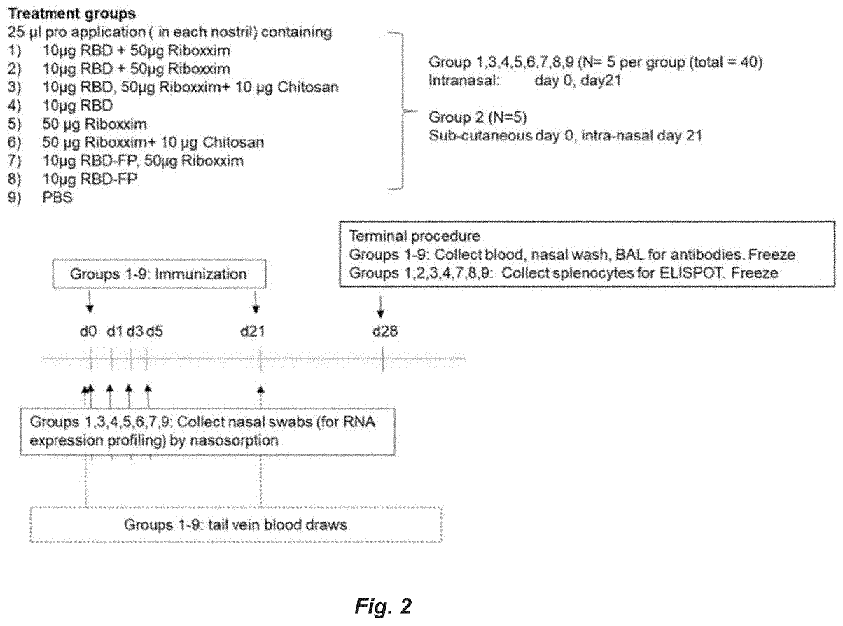

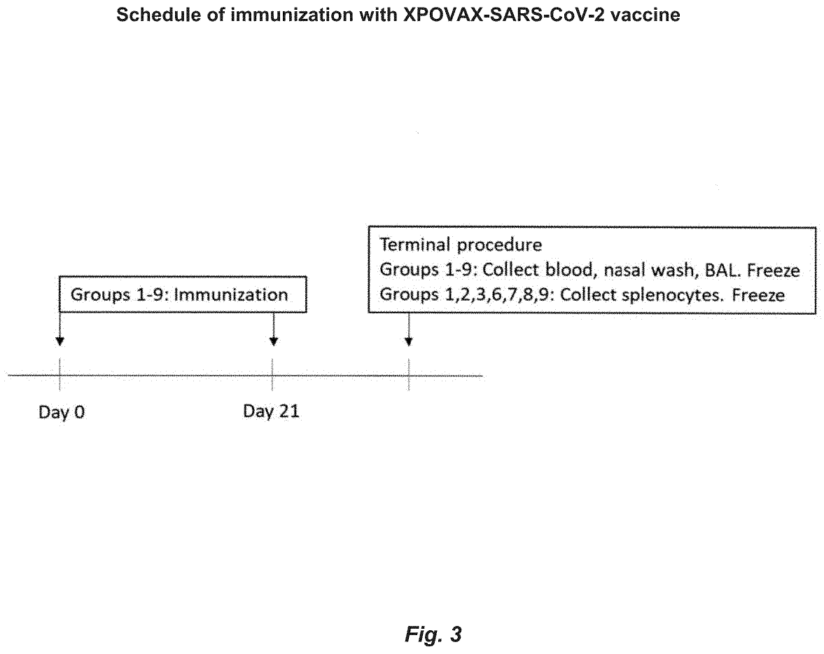

FIG. 2 shows an illustration of the vaccination scheme.

### Example 3 Immune Responses of Mice Immunized with RIBOXXIM and RBD-Fragment of SARS-CoV2 Spike Protein (Designated RBD), as Well as Claudin-4 Targeted RBD-Fragment (Designated RBD-C-CPE)

Materials and Methods

XPOVAX SARS-CoV-2 Vaccine Composition

The vaccine contained SARS-CoV-2 RBD-protein or RBD-C-CPE-Protein, adjuvanted with RIBOXXIM or unadjuvanted, with or without Chitosan (see Table 3).

TABLE 3
Amount of antigen (SARS-COV-2 RBD or SARS-COV-2 RBD-C-
CPE), adjuvant (Riboxxim), and Chitosan, in different formulations of
XPOVAX-SARS-COV-2 vaccine, per one dose in 50 μl volume
(divided over two nostrils) in different treatment groups (1-9)

| | | | | | Total volume |
|---|---|---|---|---|---|
| Group | RIBOXXIM | Chitosan | RBD | RBD-C- | administered |
| # | (μg) | (μg) | (μg) | CPE (μg) | per dose (μl) |
| 1 | 20 | — | 20 | — | 50 (2 × 25) |
| 2 | 20 | — | 20 | — | 50 (2 × 25) |
| 3 | 20 | 20 | 20 | — | 50 (2 × 25) |
| 4 | — | — | 20 | — | 50 (2 × 25) |
| 5 | 20 | — | — | — | 50 (2 × 25) |
| 6 | 20 | 20 | — | — | 50 (2 × 25) |
| 7 | 20 | — | — | 20 | 50 (2 × 25) |
| 8 | — | — | — | 20 | 50 (2 × 25) |
| 9 | — | — | — | | 50 (2 × 25) |

Animals

Female Balb/c mice (n=5 per group) were immunized at 6-8 weeks of age

Route and Schedule of Immunizations

Animals from groups 1, 3, 4, 5, 6, 7, 8 and 9 received 25 μl of the vaccine or the vehicle control (PBS, Group #9) in each nostril at days 0 and 21, resulting in a total application of 50 μl per animal per immunization. Animals from group 2 received 50 μl of the vaccine subcutaneously at day 0, and 50 μl of the vaccine intranasally at day 21.

The first immunization was performed on day 0 (prime), the second immunization on day 21 (boost).

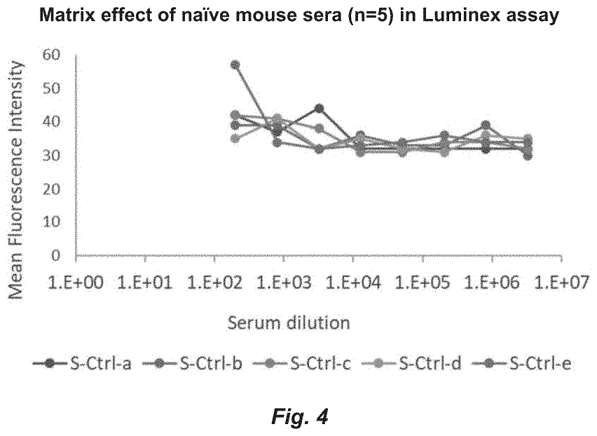

FIG. 4 shows a graphic representation of the experimental schedule.

Biological Sample Collection

Collection of blood samples was performed via the tail vein. Bronchoalveolar-lavage and collection of splenocytes was performed after sacrificing of the animals. Nasal lavages were performed at the end of the experiment. A small incision was made into the trachea using a surgical knife. A blunted 20-gauge needle was inserted through the incision and a lavage was performed by flushing 500 μl of sterilized PBS through the trachea. The nasal lavage was collected through the nostrils in a 1.5-ml micro tube. For Groups #1 to #9: Blood, BAL and nasal fluid were collected on day 0 and 28, and frozen at −20° C.

For Groups #1, 2, 3, 4, 7, 8, 9 splenocytes were collected on day 28, frozen and stored at −80°

Luminex Antibody Assays

Polyclonal antibody binding to immobilized analyte protein (recombinant SARS-CoV-2 spike, RBD, or recombinant Influenza H1-HA₁) was done in a Luminex bead-based assay according to the manufacturer's instructions (Bio-Plex, Bio-Rad Laboratories, Hercules, Calif., USA). Recombinant analyte proteins were conjugated to beads via carbodiimide chemistry. Biotinylated analytes were bound to streptavidin coated plates. The following SARS-CoV-2 spike or RBD variants were used: Wuhan-Hu-1 strain, GenBank Acc #MN908947.3. Point mutations in Wuhan RBD: Y453F, N439K, N501Y, E484K. Recombinant proteins with different labels were obtained from trenzyme GmbH (Konstanz, Germany), Sino Biological (Beiing, China) and ACROBiosystems (Newark, Del., USA). Sequence of H1-HA₁ Influenza A virus: A consensus sequence was constructed based on the sequence of A/swine/Guangxi/2499/2011(H1N1), see the following Tab. 4:

Table. 4: Consensus Protein Sequence H1N1-G4-EA HA1
1) HA1 with C-terminal His-tag (secretion signal cleaved-off in final protein) (SEQ ID NO: 1)
2) HA1-C-CPE with N-terminal His-tag (secretion signal cleaved-off in final protein) (SEQ ID NO: 2)

Tissue Culture

HEK293T cells were cultured using DMEM supplemented with 10% fetal bovine serum and 1% penicillin/streptomycin.

Pseudotype Virus Production

Production of SARS CoV 2 pseudotypes (PV) was carried out as described Di Genova et al. (2020). Briefly, HEK 293T cells were seeded for next day transfection at 50% confluence. On the day of transfection, media was replaced with complete DMEM. Plasmids used for transfection were 1000 ng pCAGGS-SARS CoV 2 Spike (Wuhan, B.1.1.7 or B.1.351), 1500 ng lentiviral vector expressing firefly luciferase pCSFLW, and 1000 ng second generation lentiviral packaging plasmid p8.91 expressing gag, pol and rev, were mixed in 200 μL optimem for 5 minutes, followed by addition of FuGENE HD at a ratio of 1:3 (DNA:FuGENE HD), and 15 min incubation at RT before adding the transfection mix to the cells. PVs were harvested after 48 hours by filtration using a 0.45 μm cellulose acetate filter.

PV representing two “variants of concern (VoC)” were generated with the spike sequences for SARS-CoV2 strains B.1.1.7 (South African variant, SA) and B.1.351 (British variant, UK), according to Di Genova et a. (2020).

Pseudotype Titration and Neutralization

The day prior to titration or neutralization, target HEK293T cells were transfected using plasmids expressing ACE2 (pcDNA3.1+) and TRSSMP2 (pCAGGS). For titration, 100 μL of harvested pseudotyped virus was added in the first row of a 96 white well plate, followed by 50 μL of DMEM to all other wells. A 2-fold serial dilution was carried out. Target cells were then added at a density of 10,000 cells per well, and plates were returned to the incubator. 48 hours later, media was aspirated and Bright-Glo (Promega) was added to the wells, incubated at room temperature for 5 minutes, and then luciferase expression levels were quantified using a GloMax Navigator microplate luminometer.

For neutralization, target cells were transfected as described above. Patient sera was serially diluted 2-fold in complete DMEM starting at a 1:10 dilution in a 96 white well plate and mixed with SARS CoV 2 pseudotypes, for 1 hour at 37° C., 5% CO2. Target cells were then added at a density of 10,000 cells per well, and plates were returned to the incubator for 48 hours prior to assaying luciferase expression levels. Data analysis to derive half-maximal inhibitory concentrations (IC50s) was carried out using GraphPad Prism 8 software. FIG. 4 shows neutralization of PV with sera and BAL from XPOVAX-immunized mice.

ELISpot Assay

IFNγ production of OVA-specific cells was analyzed using a commercially available mouse ELISPOT antibody pair according to the manufacturer's protocol (#551881, BD Biosciences). Briefly, splenocytes of immunized mice were seeded at 5×105 cells per well onto pre-coated MultiScreen®HTS Filter Plates (Merck Millipore, Burlington, Mass., USA) and re-stimulated with 10 μM of the respective peptide for 20 h. After incubation with the biotinylated secondary antibody specific for IFNγ, a streptavidin-alkaline phosphatase enzyme conjugate was added. After the addition of the BCIP®/NBT substrate solution (#1911, Merck KGaA, Darmstadt, Germany), a purple precipitate is formed as spots at the sites of captured IFNγ. Automated spot analysis and quantification were performed using the ImmunoScan® analyzer and Immunospot software v.6.0.0.2 (CTL Europe GmbH, Bonn Germany)

Results

Antibody Responses Induced by the XPOVAX-SARS-CoV-2 Vaccine

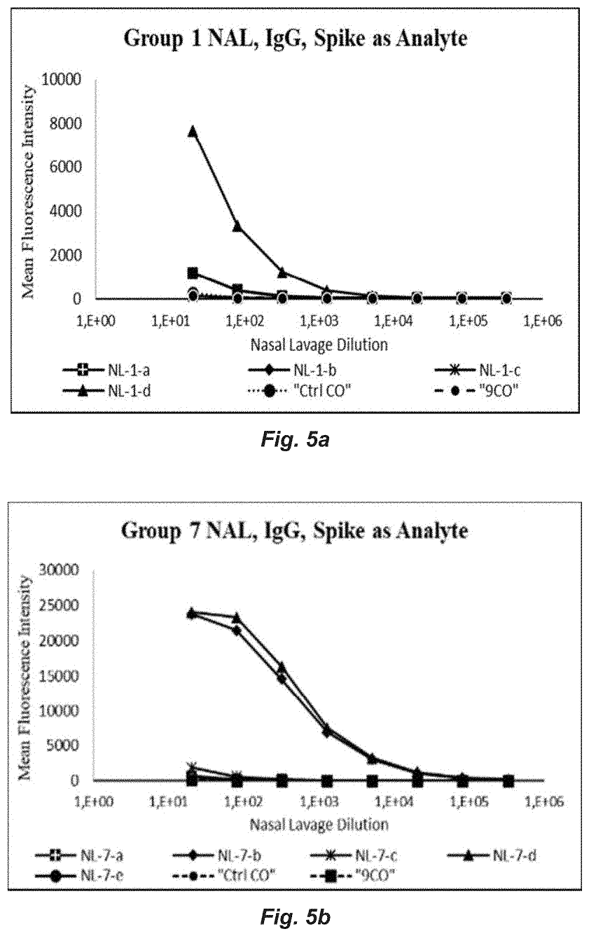

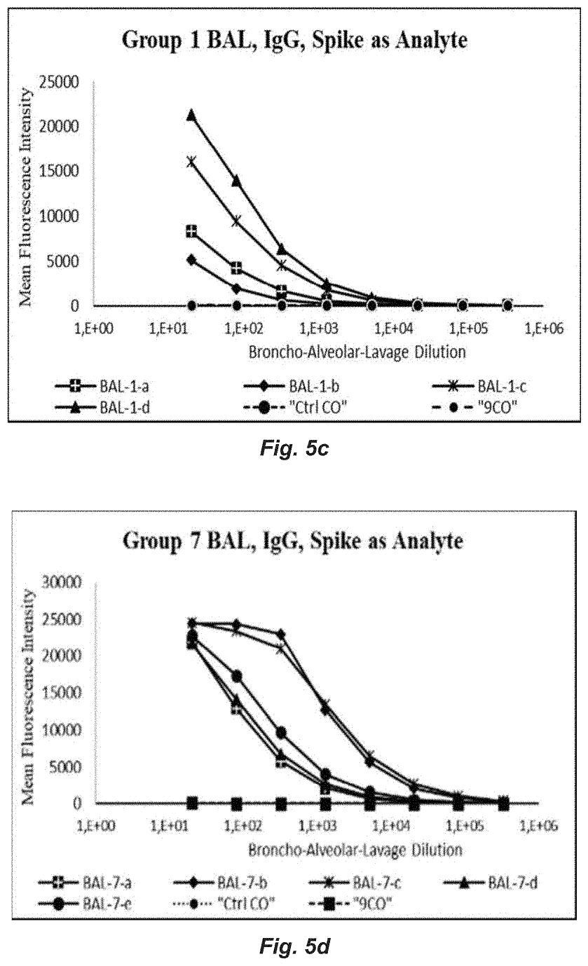

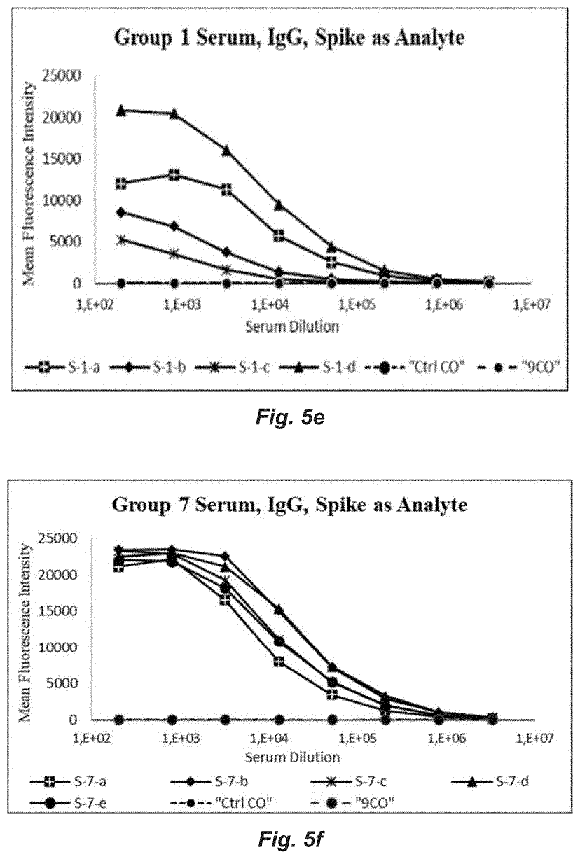

Binding antibody responses (IgG, IgA) to spike protein were measured in nasal lavage (NAL), bronchioalveolar lavage (BAL), and serum, at day 28 after immunization, using the SARS-CoV-2 spike protein (Wuhan-Hu-1) as analyte. FIG. 5 shows background reactivity of sera from control mice (group #9) immunized with PBS in the Luminex assay, and calculation of cut-off value. The cut-off value was used to determine antibody titers. No binding antibodies were detected in sera of mice immunized with vaccine containing only the RBD without adjuvant or chitosan (group 4), or animals receiving the RBD and chitosan (group 3) (data not shown).

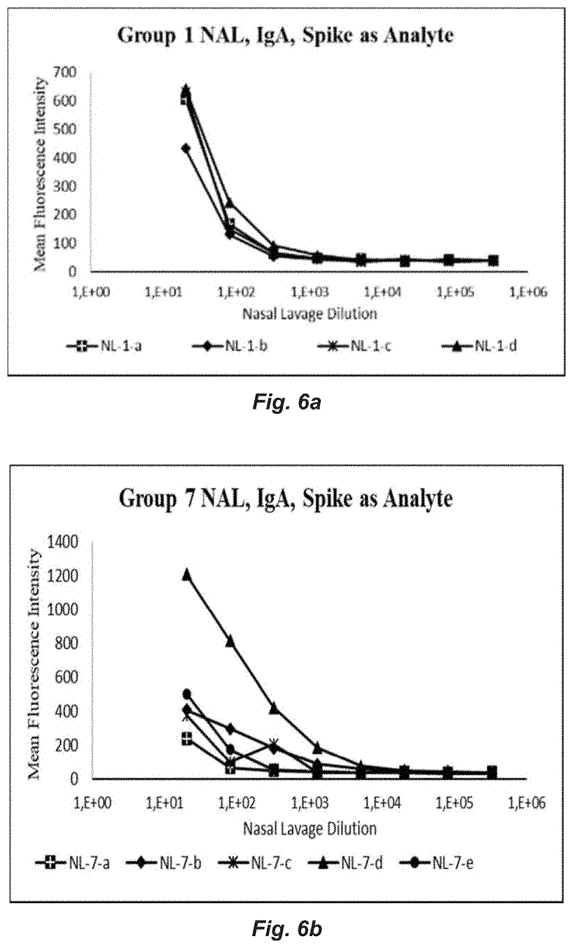

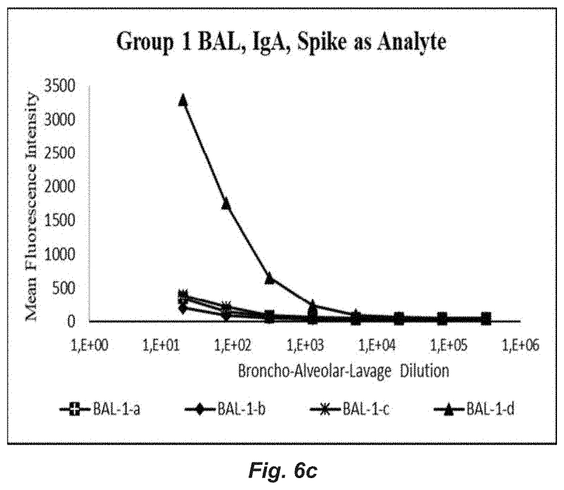

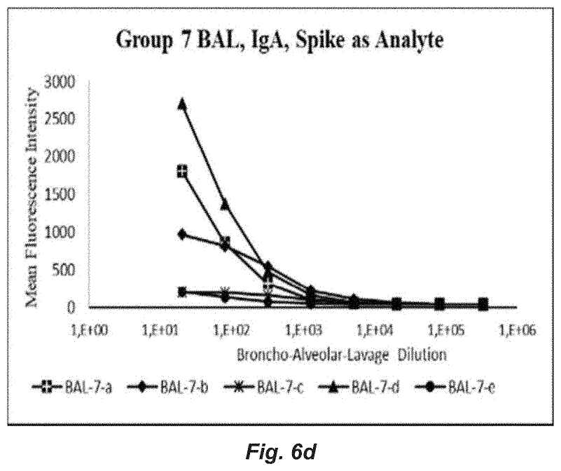

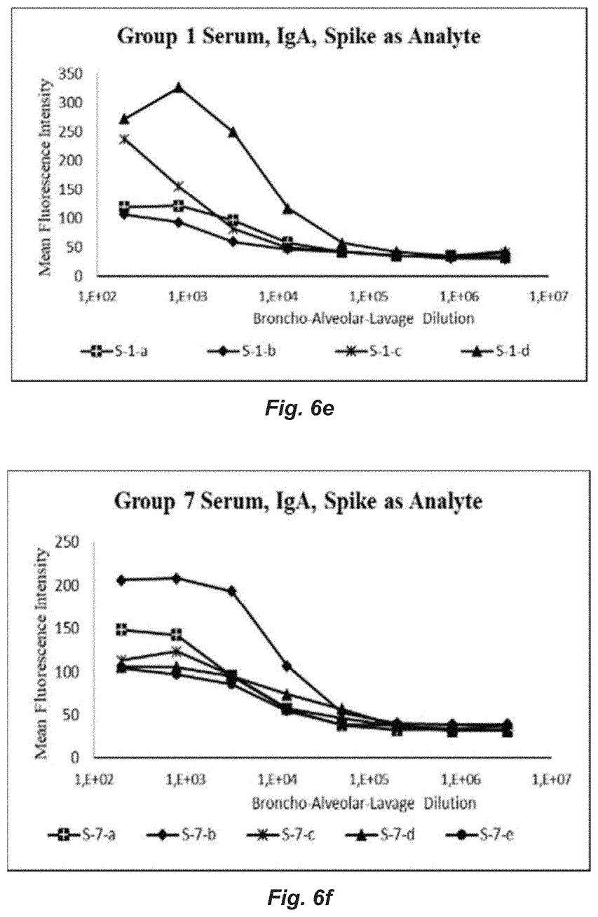

FIGS. 5a to 5e show the detection of IgG antibodies in NAL, BAL and sera of mice receiving the RBD plus Riboxxim (group #1), or RBD-C-CPE with Riboxxim (group #7). Shown are 2-fold endpoint titrations, starting at an initial serum dilution of 1:20. The RBD-C-CPE fusion protein is more immunogenic than the RBD domain alone, as shown by induction of 5.7-11.9-fold higher IgG geometric mean antibody titers in the biological fluids tested (table 3). The GMTs of IgA antibodies did not differ between groups (see FIGS. 6a to 6e and Tab. 5).

TABLE 5
Geometric mean titers of IgG and IgA antibodies induced in
NAL, BAL, and serum of mice immunized with XPOVAX-
SARS-COV-2 vaccine (groups #1 and #7 shown)

| Group | GMT | Group | GMT |
|---|---|---|---|
| #1 NAL-IgG | 320 | #1 NAL-IgA | 452 |
| #1 BAL-IgG | 14481 | #1 BAL-IgA | 2560 |
| #1 Serum-IgG | 579261 | #1 Serum-IgA | 2262 |
| #7 NAL-IgG | 3800 | #7 NAL-IgA | 970 |
| #7 BAL-IgG | 142631 | #7 BAL-IgA | 2560 |
| #7 Serum-IgG | 3276800 | #7 Serum-IgA | 4222 |

FIGS. 6a to 6f show the detection of IgA antibodies in nasal lavages, BAL and sera of mice receiving the RBD plus Riboxxim, or RBD-C-CPE with Riboxxim. Shown are 2-fold endpoint titrations, starting at an initial serum dilution of 1:20.

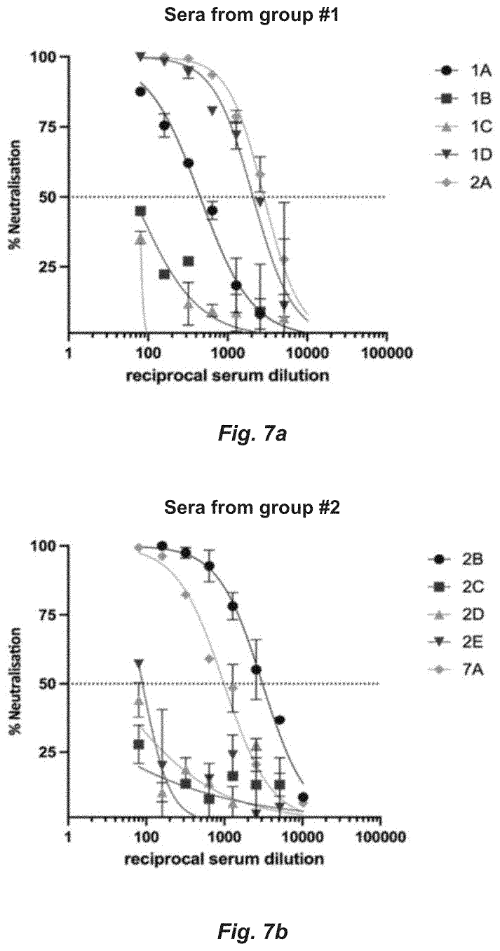

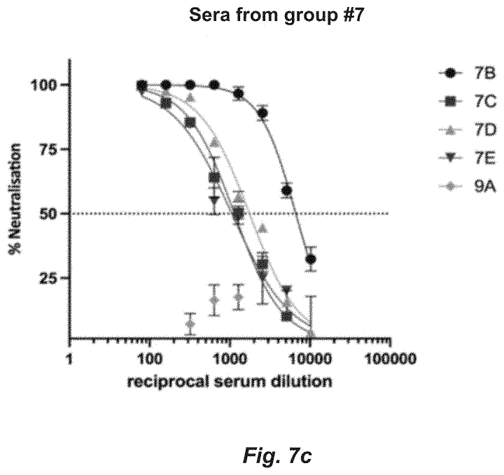

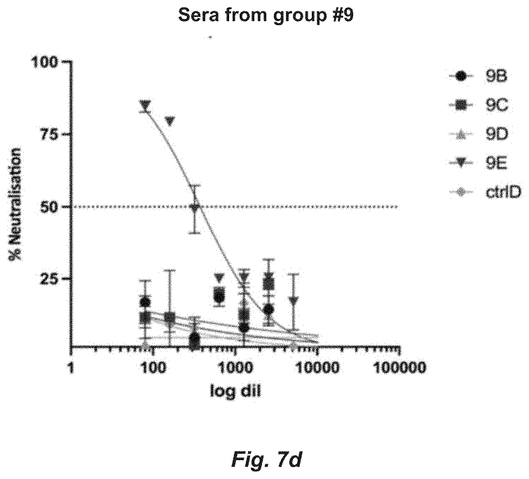

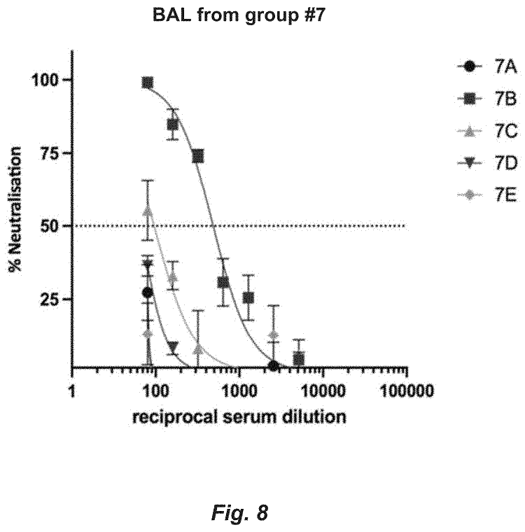

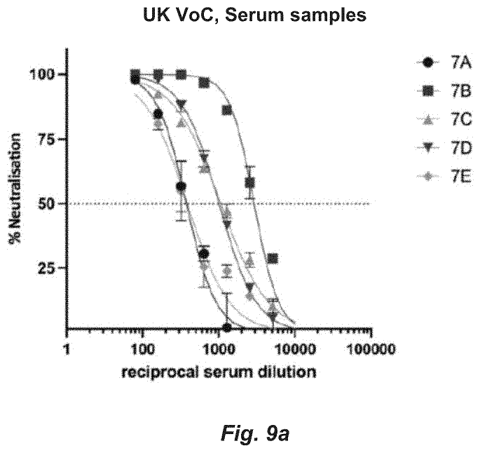

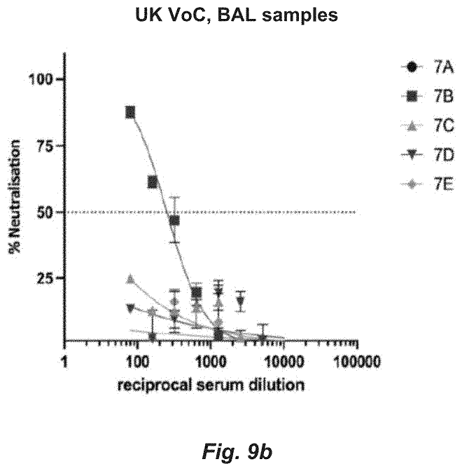

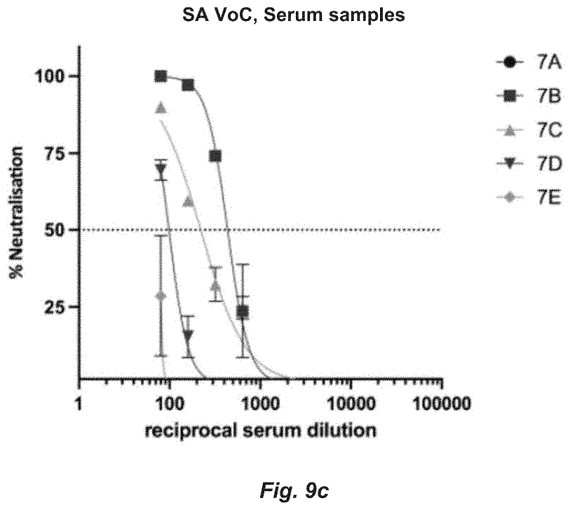

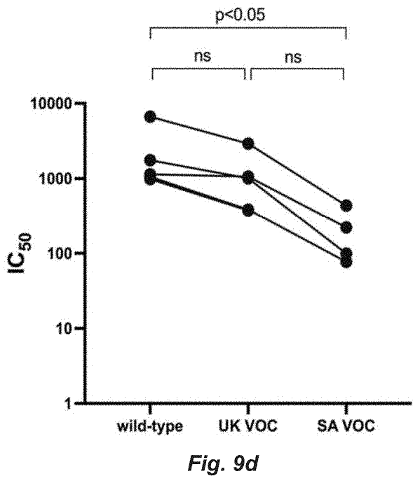

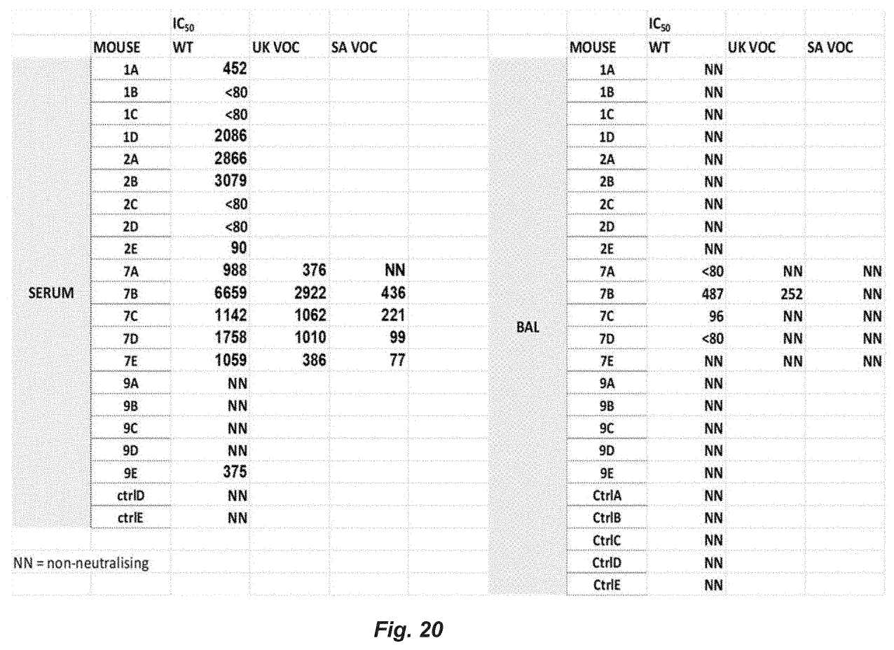

Neutralizing antibody titers were determined in sera collected on day 28 after immunization. No neutralizing antibodies were detectable in sera of mice from groups #3, 4, 5, 6, 8 and 9. Neutralizing antibodies against SARS-CoV-2 wt and VoC PV were detectable in all sera and one BAL of mice belonging to groups #1, 2, and 7, with highest titers observed in the mice which received the RBD-C-CPE antigen together with Riboxxim (see FIGS. 7 and 8). All sera with neutralizing antibodies neutralized the wildtype (wt) and UK-VoC PV with similar IC50 titers, whereas titers were approx. 10-fold lower for the SA-VoC (see FIGS. 9a, 9b, 9c and 9d, as well as FIG. 20).

T-Cell Responses

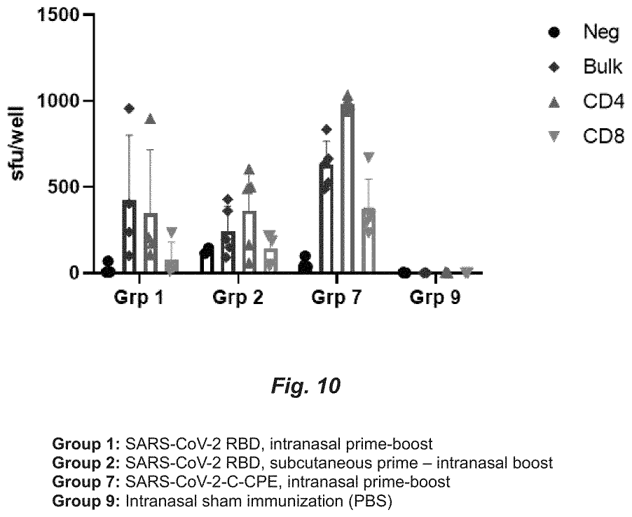

Animals in groups 1 and 2 showed RBD-specific CD4 and CD8 T-cell responses against recombinant wildtype SARS-CoV-2 RBD, which were significantly increased in group 7 that had been immunized with the RBD-C-CPE construct (FIG. 10).

### Example 4 Immune Responses of Mice Immunized with RIBOXXIM and Influenza H1-HA1 (Consensus Sequence Based on Strain H1N1 G4 EA), the Claudin-4 Targeted H1-HA1 (Designated H1-HA1-C-CPE) and the Claudin-4 Targeted RBD-Fragment of the SARS-CoV-2 Spike (Strain Wuhan-Hu-1, Designated SARS-CoV-2 RBD-C-CPE)

XPOVAX Influenza/SARS Combination Vaccine Composition

The XPOVAX Influenza vaccine contained H1-HA1 or H1-HA1-C-CPE protein, adjuvanted with Riboxxim, the Influenza/SARS-CoV-2 combination vaccine contained recombinant H1-HA1-C-CPE and SARS-CoV-2 RBD-C-CPE-Protein, adjuvanted with RIBOXXIM (Table 6).

TABLE 6
Amount of antigen (Influenza H1-HA₁, Influenza H1-HA₁-C-CPE,
SARS-CoV-2 RBD-C-CPE), adjuvant (Riboxxim), in different formulations of
XPOVAX SARS/Influenza vaccine, per one dose in 50 μl volume (divided over
two nostrils), in different treatment groups

| | | SARS- | Influenza | | | |
|---|---|---|---|---|---|---|
| Group | | CoV-2 | H1 | Influenza | | Total volume |
| # | RIBOXXIM | RBD-C- | HA₁-C- | H1 HA₁ | | administered |
| (n = 5) | (μg) | CPE (μg) | CPE (μg) | (μg) | Dosing | per dose |
| 1 | 20 | 20 | — | — | Single | 50 (2 × 25) |
| 2 | 20 | — | 20 | — | Single | 50 (2 × 25) |
| 3 | 20 | — | 20 | — | Prime- | 50 (2 × 25) |
| | | | | | boost | |
| 4 | 20 | 20 | 20 | — | Single | 50 (2 × 25) |
| 5 | 20 | 20 | 20 | — | Prime- | 50 (2 × 25) |
| | | | | | boost | |
| 6 | 20 | — | — | — | Single | 50 (2 × 25) |
| 7 | 20 | — | — | 20 | Prime- | 50 (2 × 25) |
| | | | | | boost | |

Animals

Female Balb/c mice (n=5 per group) were immunized at 6-8 weeks of age Schedule of Immunization with XPOVAX-Influenza/SARS Combination Vaccine

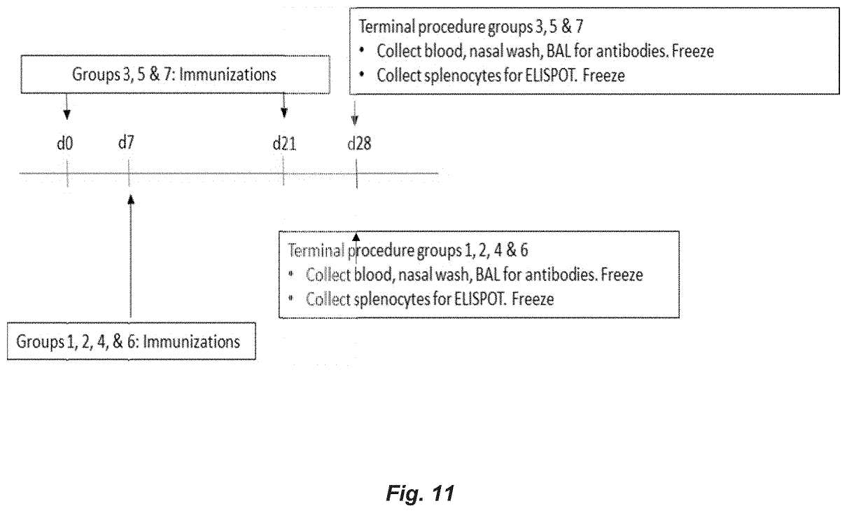

Animals from groups 1-7 received 25 μl of the vaccine in each nostril at days 0 and 21, resulting in a total application of 50 μl per animal per immunization. Animals from group 3, 5, and 7 received 50 μl of the vaccine intranasally on day 0, and 50 μl of the vaccine intranasally on day 21 (prime-boost). Animals from groups 1, 2, 4 and 6 received 50 μl of the vaccine intranasally on day 0 only (single immunization). A schematic representation of the schedule of immunization of present Example 4 is shown in FIG. 11.

Biological Sample Collection

Collection of blood samples was performed via the tail vein. Bronchoalveolar-lavage and collection of splenocytes was performed after sacrificing of the animals.

For Groups 1 to 9: Blood, BAL and nasal fluid were collected on day 28, and frozen at −20° C. For Groups 1, 2, 3, 4, 7, 8, 9 splenocytes were collected on day 28, frozen stored at −80° C.

Luminex Antibody Assays

Polyclonal antibody binding to immobilized analyte protein (SARS-CoV-2 RBD, or H1-HA1) was done in a Luminex bead-based assay according to the manufacturer's instructions (Bioplex, Biorad). Recombinant analyte proteins were conjugated to beads via carbodiimide chemistry. Biotinylated analytes were bound to streptavidin coated plates. The following SARS-CoV-2 spike or RBD variants were used: wildtype Wuhan-Hu-1 strain (GenBank: MN908947.3). RBD point mutations: Y453F, N439K, N501Y, E484K. For the influenza H1-HA1 construct a consensus sequence based on the Influenza A virus strain A/swine/Guangxi/3843/2011 was constructed (table 2). Recombinant proteins with different tags were purchased from trenzyme GmbH (Konstanz, Germany), Sino Biological (Beijing, China) and ACROBiosystems (Newark, Del., USA).

Results

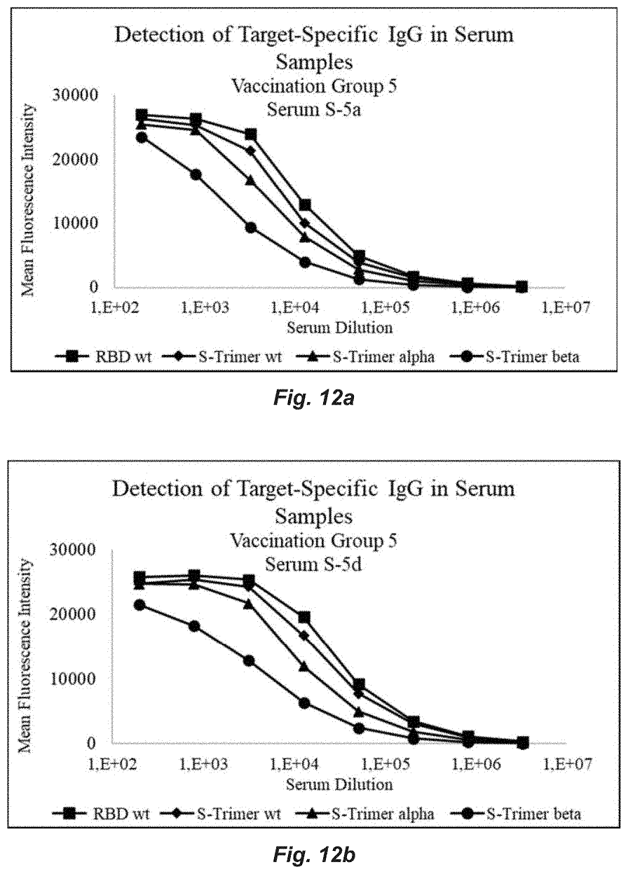

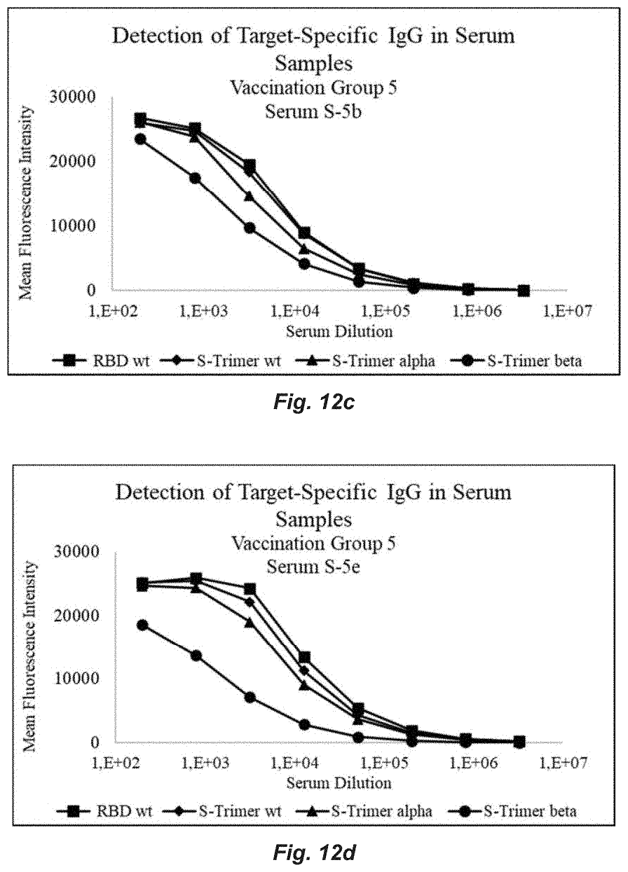

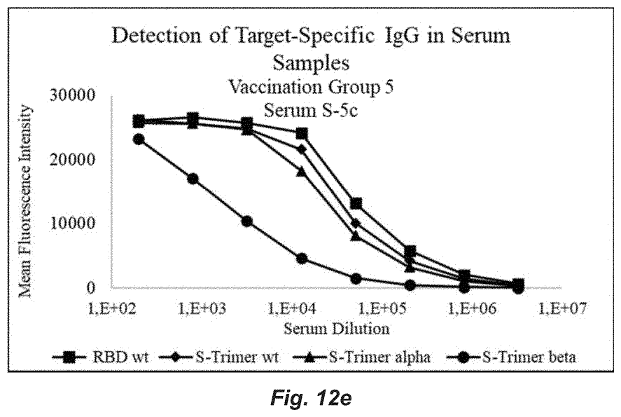

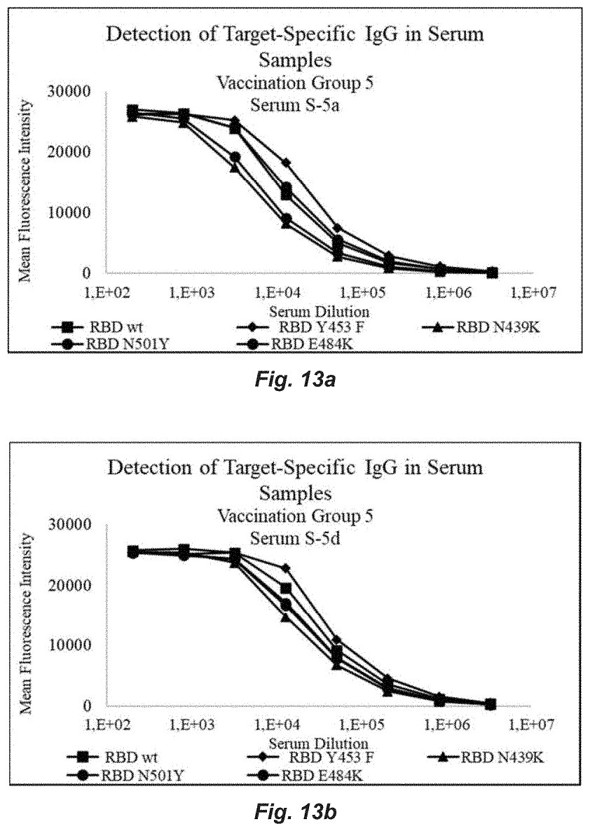

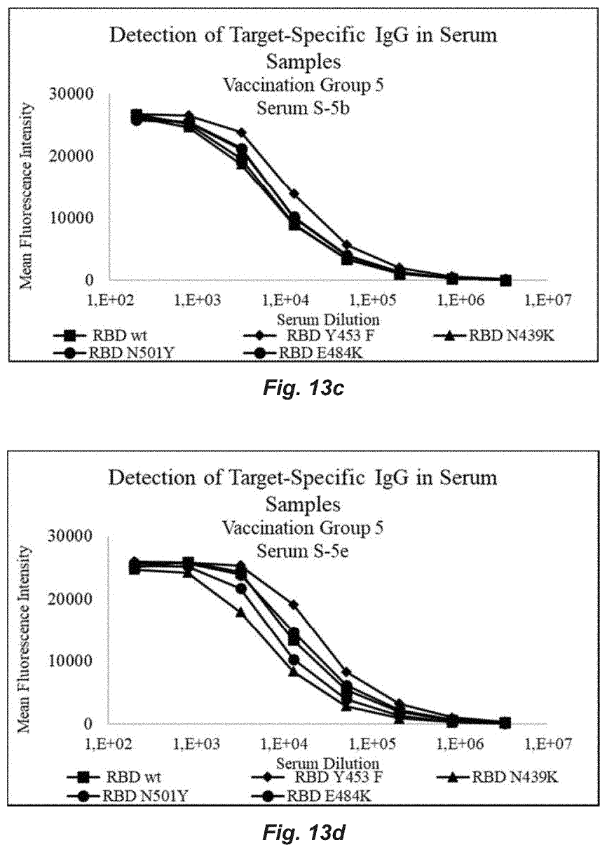

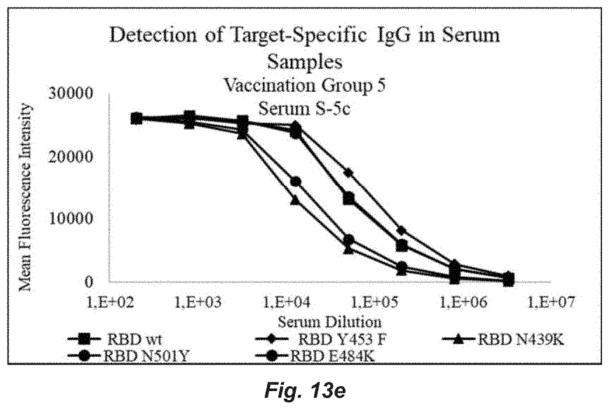

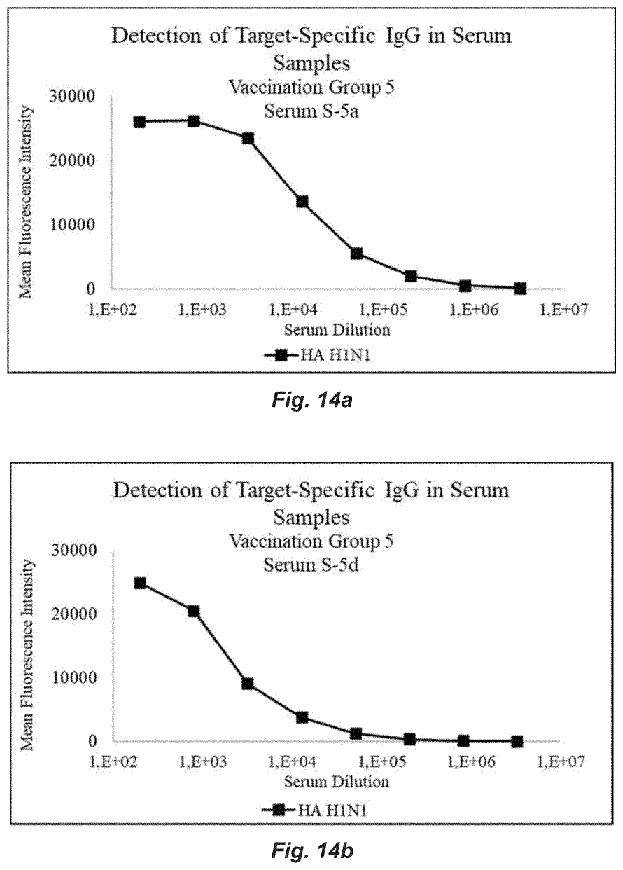

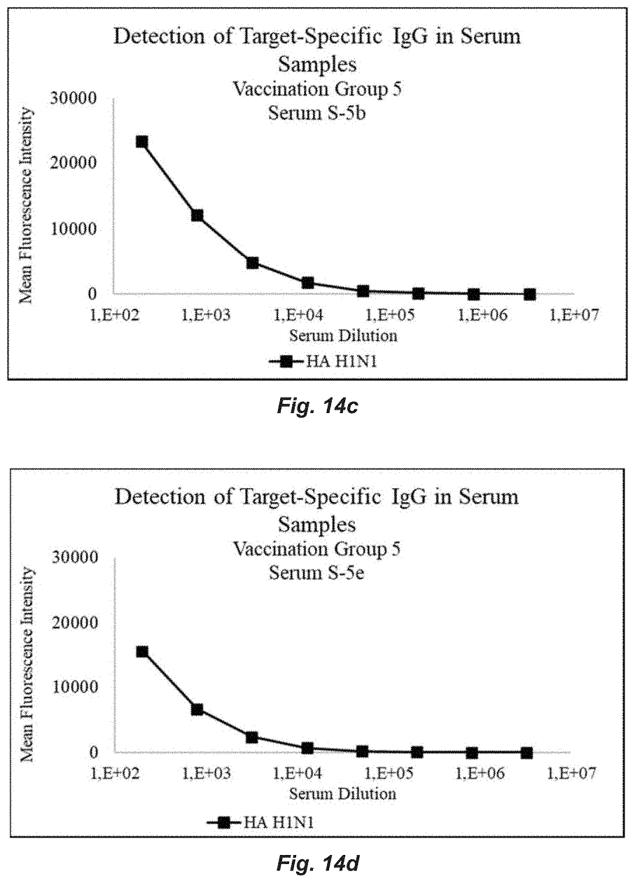

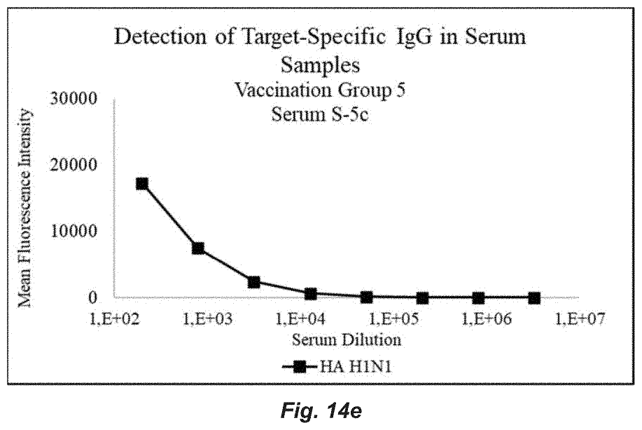

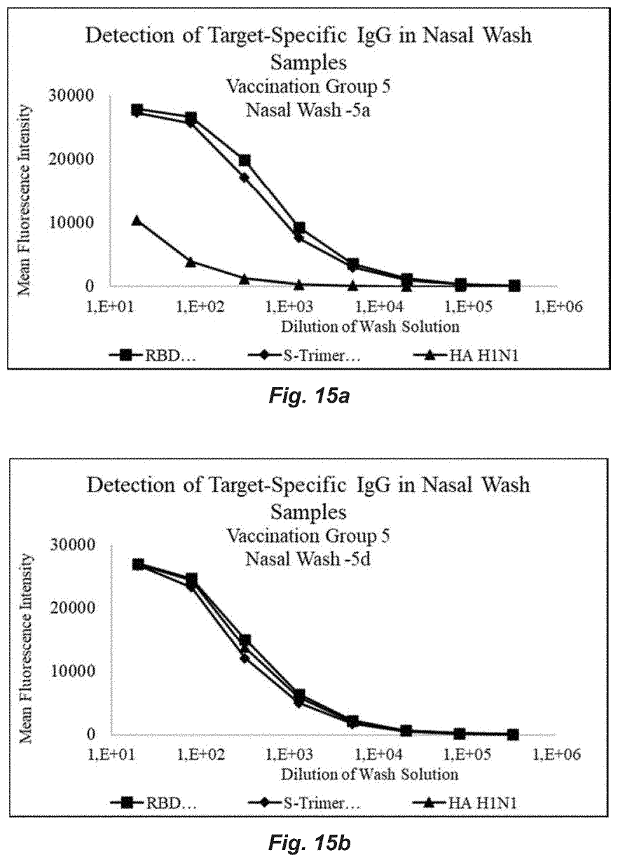

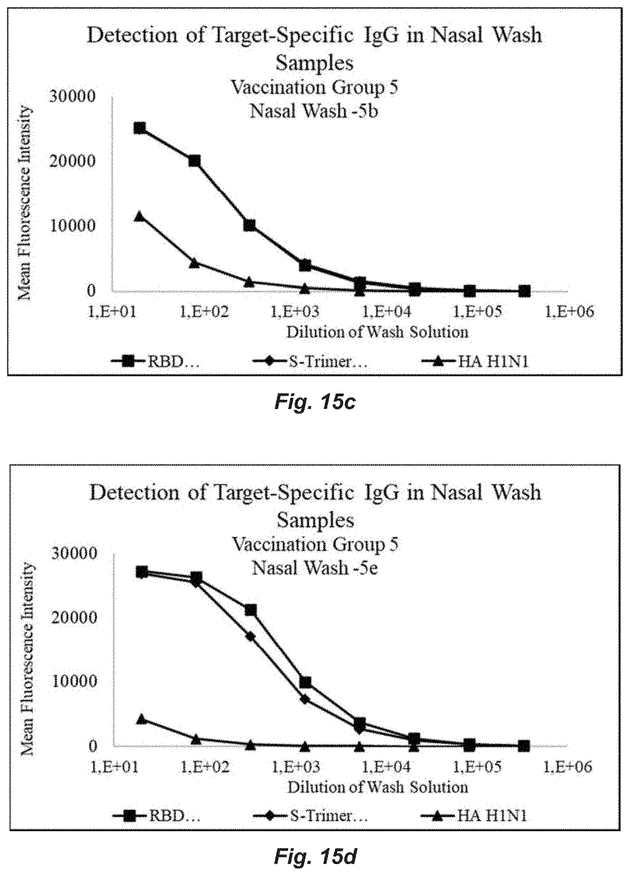

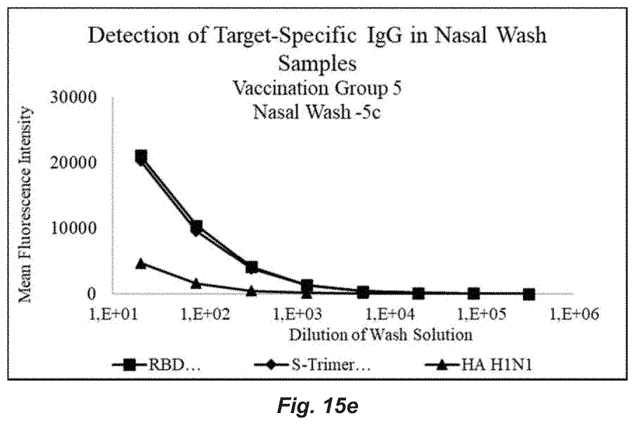

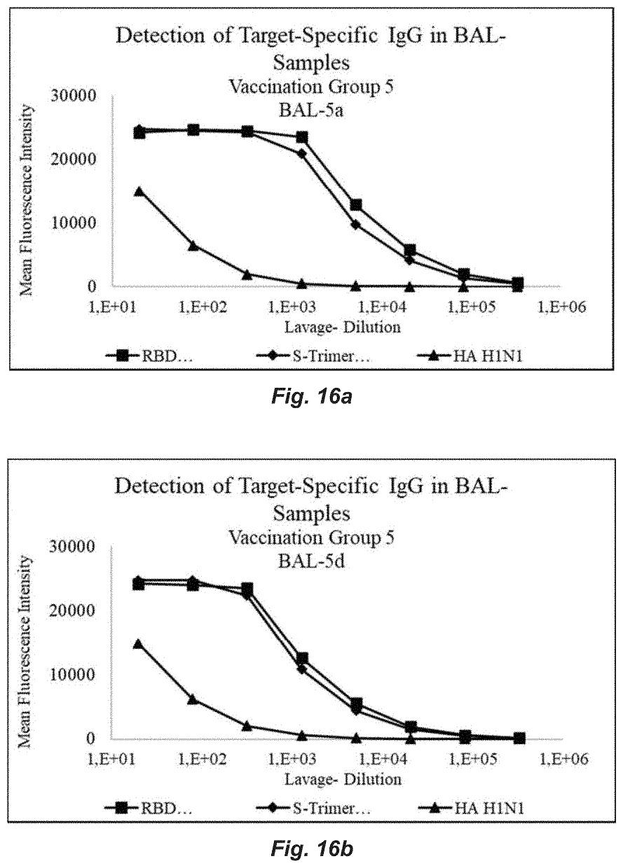

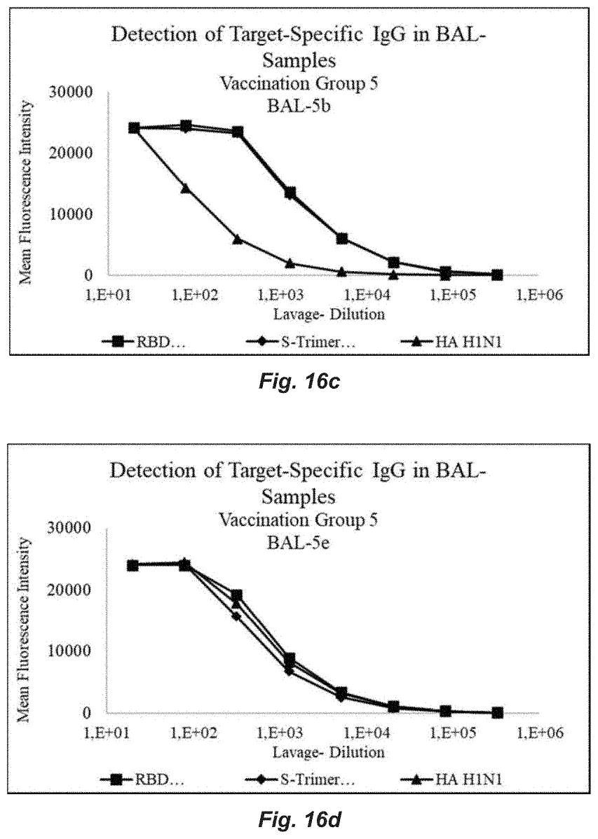

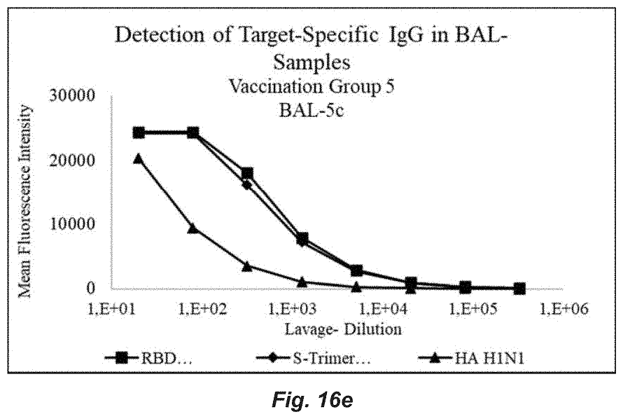

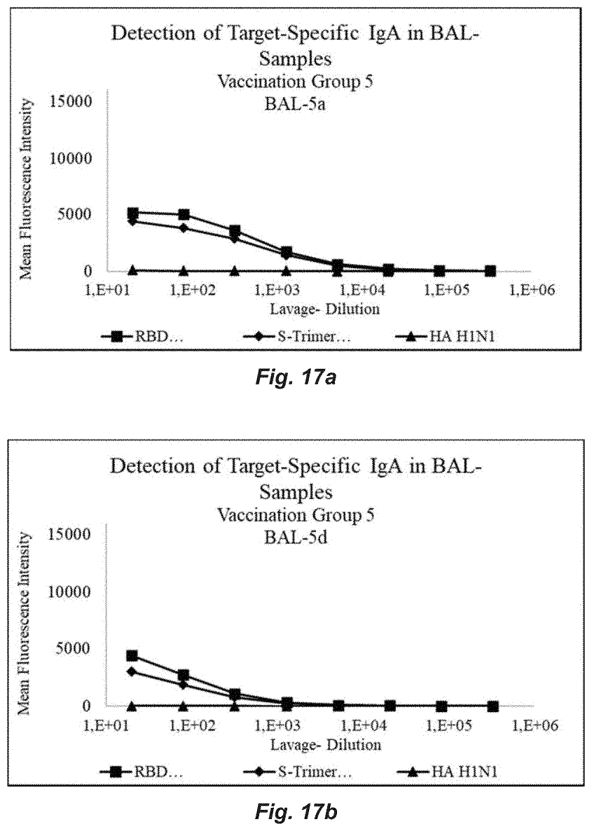

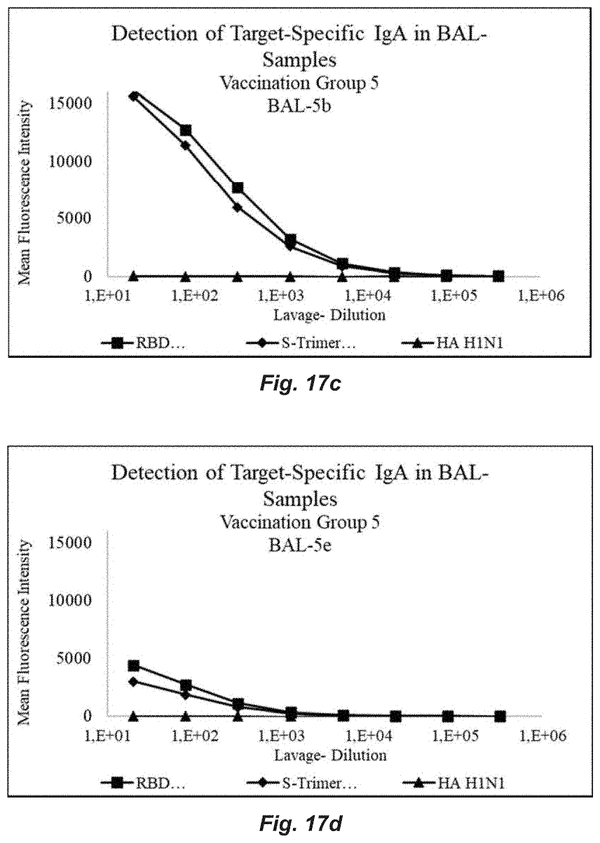

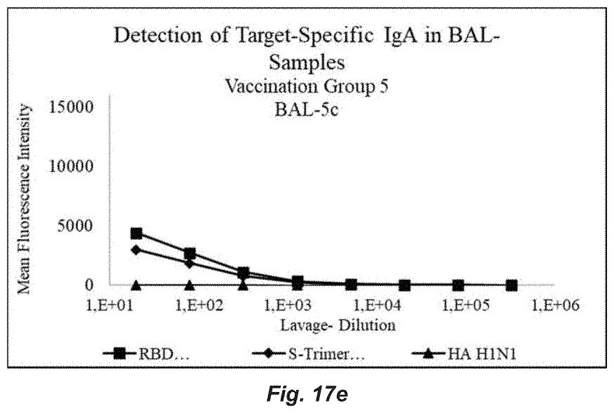

Binding antibodies were measured in mouse sera collected on day 28 post immunization. Animals immunized with the XPOVAX Influenza/SARS combination vaccine in a prime-boost regimen (group 5) had high IgG titers detectable against the SARS-CoV-2 spike and RBD, and against Influenza H1-HA1, in serum (FIGS. 12 to 14), nasal lavage (FIG. 15) and bronchio-alveolar lavage (FIG. 16), as measured by Luminex. Lower IgA titers were also detectable in bronchio-aveolar lavage (FIG. 17).

Pseudovirus Neutralization Assay (SARS-CoV-2 Strain Wuhan-Hu-1)

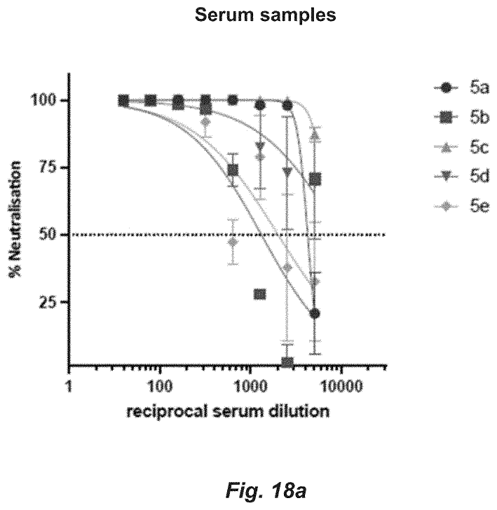

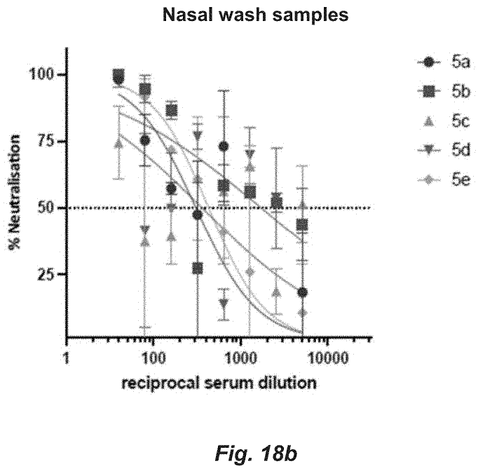

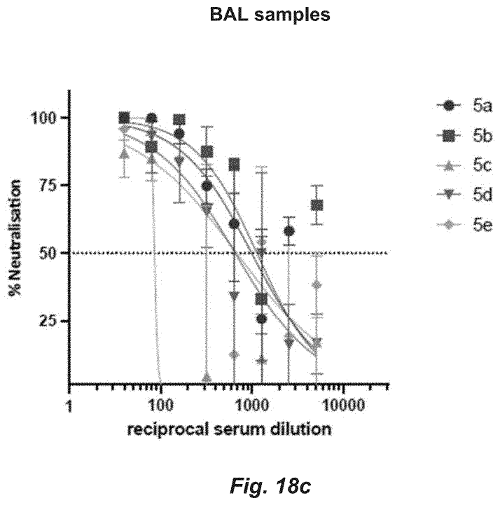

Neutralizing antibodies were detected against SARS-CoV-2 in sera, NAL and BAL of mice from group #5 (see FIG. 18).

ELISpot Assay

IFNγ production of OVA-specific cells was analyzed using a commercially available mouse ELISPOT antibody pair according to the manufacturer's protocol (#551881, BD Biosciences). Briefly, splenocytes of immunized mice were seeded at 5×105 cells per well onto pre-coated MultiScreen®HTS Filter Plates (Merck Millipore) and re-stimulated with 10 μM of the respective peptide for 20 h. After incubation with the biotinylated secondary antibody specific for IFNγ, a streptavidin-alkaline phosphatase enzyme conjugate was added. After the addition of the BCIP®/NBT substrate solution (#1911, Merck), a purple precipitate is formed as spots at the sites of captured IFNγ. Automated spot analysis and quantification were performed using the ImmunoScan® analyzer and Immunospot software v.6.0.0.2 (CTL Europe, Germany)

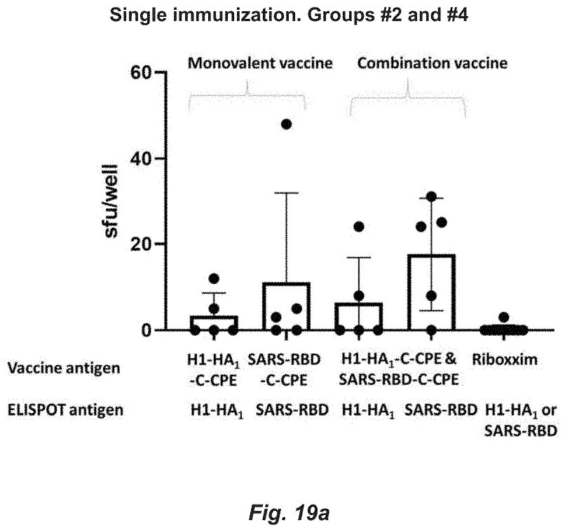

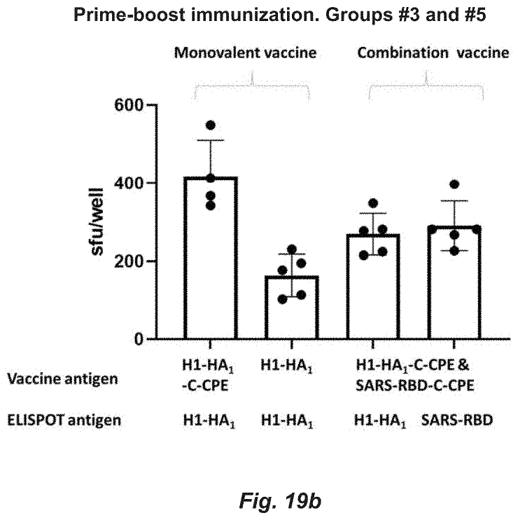

Animals immunized with the XPOVAX Influenza/SARS-CoV-2 combination vaccine (group 5) had robust T-cell responses against both immunization antigens (see FIG. 19).

Animal immunized with a single dose of the H1-HA1 antigen also showed robust T-cell responses against the homologous antigen (cf. FIG. 21).

### REFERENCES

Bolles M, Deming D, Long K, et al. A double-inactivated severe acute respiratory syndrome coronavirus vaccine provides incomplete protection in mice and induces increased eosinophilic proinflammatory pulmonary response upon challenge. J Virol. 2011; 85(23):12201-12215
Corbett K S, Edwards D, Leis S R et al. tSARS-CoV-2 mRNA vaccine development enabled by prototype pathogen preparedness. Preprint at bioRxiv, Jun. 11, 2020. doi.org/10.1101/2020.06.11.145920 (2020)
Di Genova, Cecilia; Sampson, Alex; Scott, Simon; Cantoni, Diego; Mayora-Neto, Martin; Bentley, Emma; et al. (2020): Production, titration, neutralisation and storage of SARS-CoV-2 lentiviral pseudotypes. figshare https://doi.org/10.6084/m9.figshare.13502580.v2
DiNapoli J M, Kotelkin A, Yang L, et al. Newcastle disease virus, a host range-restricted virus, as a vaccine vector for intranasal immunization against emerging pathogens. Proc Natl Acad Sci USA. 2007; 104(23):9788-9793. doi:10.1073/pnas.0703584104
Du L, Zhao G, Lin Y, et al. Intranasal vaccination of recombinant adeno-associated virus encoding receptor-binding domain of severe acute respiratory syndrome coronavirus (SARS-CoV) spike protein induces strong mucosal immune responses and provides long-term protection against SARS-CoV infection. J Immunol. 2008; 180(2):948-956. doi:10.4049/jimmunol.180.2.948
Gallagher T, Lipsitch M. Postexposure Effects of Vaccines on Infectious Diseases. Epidemiol Rev. 2019; 41(1):13-27. doi:10.1093/epirev/mxz014
Gates B. Responding to Covid-19—A Once-in-a-Century Pandemic?. N Engl J Med. 2020; 382(18):1677-1679. doi:10.1056/NEJMp2003762
He X, Lau E H Y, Wu P, et al. Temporal dynamics in viral shedding and transmissibility of COVID-19. Nat Med 2020; 26(5):672-675, doi:10.1038/s41591-020-0869-5
Hou Y J, Okuda K, Edwards C E, et al. SARS-CoV-2 Reverse Genetics Reveals a Variable Infection Gradient in the Respiratory Tract [published online ahead of print, 2020 May 27]. Cell. 2020; S0092-8674(20)30675-9. doi:10.1016/j.cell.2020.05.042
Ichinohe T, Ainai A, Ami Y, et al. Intranasal administration of adjuvant-combined vaccine protects monkeys from challenge with the highly pathogenic influenza A H5N1 virus. J Med Virol. 2010; 82(10):1754-1761. doi:10.1002/jmv.21824
Jaimes J A, Millet J K, Whittaker G R. Proteolytic Cleavage of the SARS-CoV-2 Spike Protein and the Role of the Novel S1/S2 Site. iScience. 2020; 23(6):101212. doi:10.1016/j.isci.2020.101212
Jia W, Channappanavar R, Zhang C, et al. Single intranasal immunization with chimpanzee adenovirus-based vaccine induces sustained and protective immunity against MERS-CoV infection. Emerg Microbes Infect. 2019; 8(1):760-772. doi:10.1080/22221751.2019.1620083
Kaiser T J, Smiley R A, Fergen B, Eichmeyer M, Genzow M. Influenza A virus shedding reduction observed at 12 weeks post-vaccination when newborn pigs are administered live-attenuated influenza virus vaccine. Influenza Other Respir Viruses. 2019; 13(3):274-278. doi:10.1111/irv.12630
Kakutani H, Kondoh M, Fukasaka M, Suzuki H, Hamakubo T, Yagi K. Mucosal vaccination using claudin-4-targeting. Biomaterials. 2010; 31(20):5463-71. doi: 10.1016/j.biomaterials.2010.03.047
Kanaya T, Hase K, Takahashi D, Fukuda S, Hoshino K, Sasaki I, et al. The Ets transcription factor Spi-B is essential for the differentiation of intestinal microfold cells. Nat Immunol 2012; 13:729-36. https://doi.org/10.1038/ni.2352.
Kelsall B L, Strober W. Distinct populations of dendritic cells are present in the subepithelial dome and T cell regions of the murine Peyerâ€TMs patch. J Exp Med 1996; 183:237-47.
Kim M H, Kim H J, Chang J. Superior immune responses induced by intranasal immunization with recombinant adenovirus-based vaccine expressing full-length Spike protein of Middle East respiratory syndrome coronavirus. PLoS One. 2019; 14(7):e0220196. Published 2019 Jul. 22. doi:10.1371/journal.pone.0220196
Kiyono H, Azegami T. The mucosal immune system: From dentistry to vaccine development. Proc Jpn Acad Ser B 2015; 91:423-39. https://doi.org/10.2183/pjab.91.423.
Kunisawa J, Kurashima Y, Kiyono H. Gut-associated lymphoid tissues for the development of oral vaccines. Adv Drug Deliv Rev 2012; 64:523-30. https://doi.org/10.1016/j.addr.2011.07.003.
Kunisawa J, Nochi T, Kiyono H. Immunological commonalities and distinctions between airway and digestive immunity. Trends Immunol 2008; 29: 505e13.
Lewis D J, Huo Z, Barnett S, et al. Transient facial nerve paralysis (Bell's palsy) following intranasal delivery of a genetically detoxified mutant of Escherichia coli heat labile toxin. PLoS One. 2009; 4(9):e6999. Published 2009 Sep. 16. doi:10.1371/journal.pone.0006999
Li Y, Qian H, Hang J et al. Evidence for probable aerosol transmission of SARS-CoV-2 in a poorly ventilated restaurant. Preprint at bioRxiv, Apr. 22, 2020, doi: https://doi.ord/10.1101/2020.04.16.20067728
Li K, Li Z, Wohlford-Lenane C, et al. Single-Dose, Intranasal Immunization with Recombinant Parainfluenza Virus 5 Expressing Middle East Respiratory Syndrome Coronavirus (MERS-CoV) Spike Protein Protects Mice from Fatal MERS-CoV Infection. mBio. 2020; 11(2):e00554-20. Published 2020 Apr. 7. doi:10.1128/mBio.00554-20
Lo D, Tynan W, Dickerson J, Scharf M, Cooper J, Byrne D, Brayden D, Higgins L, Evans C, O'Mahony D J: Cell culture modeling of specialized tissue: identification of genes expressed specifically by follicle-associated epithelium of Peyer's patch by expression profiling of Caco-2/Raji cocultures. Int Immunol 2004, 16:91-99.
Ma C, Li Y, Wang L, et al. Intranasal vaccination with recombinant receptor-binding domain of MERS-CoV spike protein induces much stronger local mucosal immune responses than subcutaneous immunization: Implication for designing novel mucosal MERS vaccines. Vaccine. 2014; 32(18):2100-2108. doi:10.1016/j.vaccine.2014.02.004
Lu B, Huang Y, Huang L, et al. Effect of mucosal and systemic immunization with virus-like particles of severe acute respiratory syndrome coronavirus in mice. Immunology. 2010; 130(2):254-261. doi:10.1111/j.1365-2567.2010.03231.x
Malcolm B A, Aerts C A, Dubois K J, et al. PrEP-001 prophylactic effect against rhinovirus and influenza virus—RESULTS of 2 randomized trials. Antiviral Res. 2018; 153:70-77. doi:10.1016/j.antiviral.2018.03.005
Maroof A, Yorgensen Y M, Li Y, Evans J T. Intranasal vaccination promotes detrimental Th17-mediated immunity against influenza infection. PLoS Pathog. 2014; 10(1):e1003875. doi:10.1371/journal.ppat.1003875
Moghadas S M, Bowman C S, Röst G, Fisman D N, Wu J. Post-exposure prophylaxis during pandemic outbreaks. BMC Med. 2009; 7:73. Published 2009 Dec. 2. doi:10.1186/1741-7015-7-73
Monto A S, Fukuda K. Lessons From Influenza Pandemics of the Last 100 Years. Clin Infect Dis. 2020; 70(5):951-957. doi:10.1093/cid/ciz803
Mora J R, Iwata M, Eksteen B, Song S-Y, Junt T, Senman B, et al. Generation of gut-homing IgA-secreting B cells by intestinal dendritic cells. Science 2006; 314:1157-60. https://doi.org/10.1126/science.1132742.
Mutsch M, Zhou W, Rhodes P, et al. Use of the inactivated intranasal influenza vaccine and the risk of Bell's palsy in Switzerland. N Engl J Med. 2004; 350(9):896-903. doi:10.1056/NEJMoa030595
Overton E T, Goepfert P A, Cunningham P, et al. Intranasal seasonal influenza vaccine and a TLR-3 agonist, rintatolimod, induced cross-reactive IgA antibody formation against avian H5N1 and H7N9 influenza H A in humans. Vaccine. 2014; 32(42):5490-5495. doi:10.1016/j.vaccine.2014.07.078
Pujadas E, Chaudhry F, McBride R et al. SARS-CoV-2 Viral Load Predicts COVID-19 Mortality. Preprint at medRxiv, Jun. 12, 2020. https://doi.org/10.1101/2020.06.11.20128934
Robbiani D F, Gaebler C, Muecksch F, et al. Convergent antibody responses to SARS-CoV-2 in convalescent individuals [published online ahead of print, 2020 Jun. 18]. Nature. 2020; 10.1038/s41586-020-2456-9
Roper R L, Rehm K E. SARS vaccines: where are we?Expert Rev Vaccines. 2009; 8(7):887-898. doi:10.1586/erv.09.43
Sato S, Kaneto S, Shibata N, Takahashi Y, Okura H, Yuki Y, et al. Transcription factor Spi-B-dependent and -independent pathways for the development of Peyer's patch M cells. Mucosal Immunol 2013; 6:838-46. https://doi.org/10.1038/mi.2012.122.
Seibert C W, Rahmat S, Krause J C, et al. Recombinant IgA is sufficient to prevent influenza virus transmission in guinea pigs. J Virol. 2013; 87(14):7793-7804. doi:10.1128/JVI.00979-13
Shannon I, White C L, Nayak J L. Understanding Immunity in Children Vaccinated With Live Attenuated Influenza Vaccine. J Pediatric Infect Dis Soc. 2020; 9(Supplement_1):S10-S14. doi:10.1093/jpids/piz083
Suzuki H, Watari A, Hashimoto E, et al. C-Terminal Clostridium perfringens Enterotoxin-Mediated Antigen Delivery for Nasal Pneumococcal Vaccine. PLoS One. 2015; 10(5):e0126352. Published 2015 May 27. doi:10.1371/journal.pone.0126352
Takaki H, Kure S, Oshiumi H, et al. Toll-like receptor 3 in nasal CD103+ dendritic cells is involved in immunoglobulin A production. Mucosal Immunol. 2018; 11(1):82-96. doi:10.1038/mi.2017.48
Tay M Z, Poh C M, Rénia L, MacAry P A, Ng LFP. The trinity of COVID-19: immunity, inflammation and intervention. Nat Rev Immunol. 2020; 20(6):363-374. doi:10.1038/s41577-020-0311-8
ter Meulen J, van den Brink E N, Poon L L, et al. Human monoclonal antibody combination against SARS coronavirus: synergy and coverage of escape mutants. PLoS Med. 2006; 3(7):e237. doi:10.1371/journal.pmed.0030237
Treanor J J, Betts R F, Erb S M, Roth F K, Dolin R. Intranasally administered interferon as prophylaxis against experimentally induced influenza A virus infection in humans. J Infect Dis. 1987; 156(2):379-383.
Tseng C T, Sbrana E, Iwata-Yoshikawa N, et al. Immunization with SARS coronavirus vaccines leads to pulmonary immunopathology on challenge with the SARS virus [published correction appears in PLoS One. 2012; 7(8). doi:10.1371/annotation/2965cfae-b77d-4014-8b7b-236e01a35492].
Vabret N, Britton G J, Gruber C, et al. Immunology of COVID-19: Current State of the Science. Immunity. 2020; 52(6):910-941. doi:10.1016/j.immuni.2020.05.002
van Doremalen N, Lambe T, Spencer A et al. ChAdOx1 nCoV-19 vaccination prevents SARS-CoV-2 1 pneumonia in rhesus macaques. Preprint at bioRxiv, 2020 May 13:2020.05.13.093195. doi: 10.1101/2020.05.13.093195
Wang J, Lopez-Fraga M, Rynko A, Lo D D: TNFR and LTbetaR agonists induce follicle-associated epithelium and M cell specific genes in rat and human intestinal epithelial cells. Cytokine 2009, 47:69-76.
Wang Q, Zhang L, Kuwahara K, et al. Immunodominant SARS Coronavirus Epitopes in Humans Elicited both Enhancing and Neutralizing Effects on Infection in Non-human Primates [published correction appears in ACS Infect Dis. 2020 May 8; 6(5):1284-1285]. ACS Infect Dis. 2016; 2(5):361-376. doi:10.1021/acsinfecdis.6b00006
Wang Y, Kang H, Liu X, Tong Z. Asymptomatic cases with SARS-CoV-2 infection [published online ahead of print, 2020 May 8]. J Med Virol. 2020; 10.1002/jmv.25990. doi:10.1002/jmv.25990
Weingartl H, Czub M, Czub S, et al. Immunization with modified vaccinia virus Ankara-based recombinant vaccine against severe acute respiratory syndrome is associated with enhanced hepatitis in ferrets. J Virol. 2004; 78(22):12672-12676. doi:10.1128/JVI.78.22.12672-12676.2004
Xu S et al. mRNA Vaccine Era—Mechanisms, Drug Platform and Clinical Prsopection. Int. J. Mol. Sci. 2020, 21, 6582 doi:10.3390/ijms21186582
Zost S J, Gilchuk P, Chen R E, et al. Rapid isolation and profiling of a diverse panel of human monoclonal antibodies targeting the SARS-CoV-2 spike protein [published online ahead of print, 2020 Jul. 10]. Nat Med. 2020; 10.1038/s41591-020-0998-x

1. A method for the prevention and/or treatment of a viral respiratory infection in a human subject comprising the step of administering, preferably by intra-nasal administration, an effective amount of a pharmaceutical composition pre-exposure or post-exposure of said subject to a virus causing said viral respiratory infection, or in a subject at least suspected to have been exposed to a virus causing said viral respiratory infection, respectively, wherein said composition comprises (i) at least one antigenic component of said virus and/or a nucleic acid encoding at least one antigenic peptide component of said virus and (ii) one or more adjuvants eliciting an innate immune response in the subject against said virus.

2. (canceled)

3. The method of claim 1 wherein the composition is self-administered by said subject.

4. The method of claim 1 wherein the composition is administered 30 seconds to 72 hours, preferably 3 min to 24 hours, more preferably 5 min to 12 hours post-exposure or post-suspected exposure to said subject, and optionally, the composition is further administered to the subject at least once daily for one week, or further administered once every 48 hours for one week, most preferably once every 72 hours for one week.

5. (canceled)

6. The method of claim 4 wherein the composition is administered at least once to the subject after completion of the optional administration as defined in claim 4, preferably at least once one week after completion of the optional administration as defined in claim 4.

7. (canceled)

8. The method of claim 1 wherein the incubation time of said viral respiratory infection is 8 days+/−2 days (95% confidence interval), preferably 5+/−1 days (95% confidence interval), most preferably 3+/−2 days (95% confidence interval).

9. (canceled)

10. The method of claim 1 wherein the at least one antigenic component of the virus is selected from the group consisting of inactivated virus, virus subunits, viral proteins, preferably viral structural proteins and/or viral non-structural proteins and/or viral enzymes, peptides comprising an epitope of a protein of said virus, and/or the nucleic acid encoding at least one antigenic peptide component of said virus is selected from the group consisting of viral proteins, preferably viral structural proteins and/or viral non-structural proteins and/or viral enzymes, and peptides comprising an epitope of a protein of said virus.

11. (canceled)

12. The method of claim 1 wherein the composition contains at least one protein or at least a peptide comprising an epitope of a protein of a virus selected from the group consisting of SARS-like coronaviruses, influenza viruses, viruses of the Paramyxoviridae family, viruses of the Pneumoviridae family and viruses of the Poxviridae family and/or at least one nucleic acid encoding a protein or at least a peptide comprising an epitope of a protein of a virus selected from the group consisting of SARS-like coronaviruses, influenza viruses, viruses of the Paramyxoviridae family, viruses of the Pneumoviridae family and viruses of the Poxviridae family.

13. The method of claim 12 wherein the SARS-like coronavirus is selected from the group consisting of SARS-coronavirus, MERS and SARS-coronavirus-2.

14. (canceled)

15. The method of claim 12 wherein the composition comprises a peptide comprising an epitope of the spike protein, preferably of the receptor binding domain (RBD), of said SARS-like coronavirus and/or a nucleic acid encoding an epitope of the spike protein preferably of the receptor binding domain (RBD), of said SARS-like coronavirus.

16. (canceled)

17. (canceled)

18. The method of claim 7 wherein the influenza virus is selected from the group consisting of H5N1, H1N1, H2N2, H3N2 and H7N1 viruses, preferably the genotype 4 (G4) Eurasian avian-like (EA) H1N1 influenza virus.

19. (canceled)

20. The method of claim 18 wherein the protein or at least a peptide comprising an epitope of said protein is selected from hemagglutinin subunits HA1 and HA2, preferably the protein or at least a peptide of said protein has an amino acid consensus sequence based on the G4 EA virus.

21. (canceled)

22. The method of claim 12 wherein the virus of the Paramyxoviridae is selected from the group consisting of Respiratory syncytial virus, Parainfluenza virus 1 to 3, Hendra virus and Nipah Virus; or wherein the virus of the Pneumoviridae family is Metapneumovirus; or wherein the virus of the Poxviridae family is selected from the group consisting of Variola major (smallpox) and monkeypox.

23. (canceled)

24. (canceled)

25. The method of claim 1 wherein at least one antigenic component is linked to a mucosa-targeting moiety, preferably C-CPE; and/or wherein the nucleic acid encodes at least one antigenic peptide component of said virus linked to a mucosa-targeting moiety, preferably C-CPE.

26. (canceled)

27. (canceled)

28. The method of claim 1 wherein the nucleic acid is an mRNA, optionally containing one or more nucleotide analogues, preferably one or more pseud-uridine nucleotides.

29. (canceled)

30. (canceled)

31. (canceled)

32. (canceled)

33. The method of claim 28 wherein the composition comprises the mRNA formulated as a lipid nanoparticle.

34. The method of claim 1 wherein one of the adjuvants or the adjuvant is a TLR-3 and/or RIG-I agonist; or wherein one of the adjuvants or the adjuvant is selected from the group consisting of TLR4, TLR7/8, TLR9 and STING agonists.

35. (canceled)

36. The method of claim 34 wherein the adjuvant is a dsRNA of at least 45 bp preferably a perfectly annealed dsRNA of at least 45 bp having two blunt ends or a perfectly annealed dsRNA having one blunt end and having a single-stranded overhang of 1 to 5 nt, preferably 1 to 3 nt, at the other end of the dsRNA.

37. (canceled)

38. The method of claim 36 wherein the dsRNA comprises a free 5′-triphophate group at least on one double-stranded end of said dsRNA.

39. The method of claim 38 wherein the dsRNA has a length of 45 to 200 bp, preferably 45 to 100 bp.

40. The method of claim 1 wherein the composition comprises at least one carrier selected from the group consisting of polyethyleneimine, PGLA, cationic liposomes, sucrose, trehalose, arginine, mannitol, histidine, sodium succinate, and mixtures of two or more thereof.

41. A pharmaceutical composition comprising (a) at least one antigenic component of a virus causing a respiratory infection, said at least one antigenic component being coupled to a mucosa-targeting moiety and/or a nucleic acid encoding at least one antigenic peptide component of a virus causing a respiratory infection linked to a mucosa-targeting moiety, and (b) one or more TLR-3 and/or RIG-I agonists.

42. The composition of claim 41 wherein the composition contains at least one protein or at least a peptide comprising an epitope of a protein of a virus selected from the group consisting of SARS-like coronaviruses, influenza viruses, viruses of the Paramyxoviridae family, viruses of the Pneumoviridae family and viruses of the Poxviridae family and/or at least one nucleic acid encoding a protein or at least a peptide comprising an epitope of a protein of a virus selected from the group consisting of SARS-like coronaviruses, influenza viruses, viruses of the Paramyxoviridae family, viruses of the Pneumoviridae family and viruses of the Poxviridae family; or wherein the composition contains a nucleic acid encoding a protein or at least a peptide comprising an epitope of a protein of a virus selected from the group consisting of SARS-like coronaviruses, influenza viruses, viruses of the Paramyxoviridae family, viruses of the Pneumoviridae family and viruses of the Poxviridae family and/or at least one nucleic acid encoding a protein or at least a peptide comprising an epitope of a protein of a virus selected from the group consisting of SARS-like coronaviruses, influenza viruses, viruses of the Paramyxoviridae family, viruses of the Pneumoviridae family and viruses of the Poxviridae family.

43. The composition of claim 42 wherein the SARS-like coronavirus is selected from the group consisting of SARS-coronavirus, MERS and SARS-coronavirus-2.

44. The composition of claim 41 wherein the at least one antigenic component is linked to a mucosa-targeting moiety, preferably C-CPE; or wherein the nucleic acid encodes at least one antigenic peptide component of said virus linked to a mucosa-targeting moiety, preferably C-CPE.

45. (canceled)

46. (canceled)

47. The composition of claim 41 wherein the nucleic acid is an mRNA, optionally containing one or more nucleotide analogues, preferably one or more pseud-uridine nucleotides.

48. (canceled)

49. The composition of claim 47 wherein the composition comprises the mRNA formulated as a lipid nanoparticle.

50. The composition of claim 41 wherein the TLR-3 agonist is a dsRNA of at least 45 bp, preferably a perfectly annealed dsRNA of at least 45 bp having two blunt ends or a perfectly annealed dsRNA having one blunt end and having a single-stranded overhang of 1 to 5 nt, preferably 1 to 3 nt, at the other end of the dsRNA, optionally comprising a free 5′-triphophate group at least on one double-stranded end of said dsRNA.

51. The composition of claim 21 comprising a carrier selected from the group consisting of polyethyleneimine, PGLA, cationic liposomes, sucrose, trehalose, arginine, mannitol, histidine, sodium succinate, and mixtures of two or more thereof.

52. The composition of claim 21 being in liquid or lyophilized form.

53. A pharmaceutical kit comprising an intra-nasal delivery device and a unit dose of the pharmaceutical composition of claim 21.


SEQUENCE LISTING (14 sequences)

SEQ ID NO: 1 - protein, 368 aa, Artificial Sequence
  HA1 with C-terminal His-tag and secretion signal
MNFGLRLIFLVLTLKGVQAMDTICVGYHANNSTDTVDTILEKNVTVTHSVNLLENSHNGKLCSLNGKIPLQLGNCNVAGWILGNPKCDLLLTANSWSYIIETSNSKNGACYPGEFADYEELKEQLSTVSSFERFEIFPKATSWPNHDTTRGTTVACSHSGANSFYRNLLWIVKKGNSYPKLSKSYTNNKGKEVLVIWGVHHPPTDSDQQTLYQNNHTYVSVGSSKYYKRFTPEIVARPKVREQAGRMNYYWTLLDQGDTITFEATGNLIAPWHAFALKKGSSSGIMRSDAQVHNCTTKCQTPHGALKGNLPFQNVHPVTIGKCPKYVKSTQLRMATGLRNIPSIQSRGENLYFQGGGSHHHHHHHHHH

SEQ ID NO: 2 - protein, 329 aa, Artificial Sequence
  SITE 1..1: optionally present
  HA1 without His-tag and without secretion signal
MDTICVGYHANNSTDTVDTILEKNVTVTHSVNLLENSHNGKLCSLNGKIPLQLGNCNVAGWILGNPKCDLLLTANSWSYIIETSNSKNGACYPGEFADYEELKEQLSTVSSFERFEIFPKATSWPNHDTTRGTTVACSHSGANSFYRNLLWIVKKGNSYPKLSKSYTNNKGKEVLVIWGVHHPPTDSDQQTLYQNNHTYVSVGSSKYYKRFTPEIVARPKVREQAGRMNYYWTLLDQGDTITFEATGNLIAPWHAFALKKGSSSGIMRSDAQVHNCTTKCQTPHGALKGNLPFQNVHPVTIGKCPKYVKSTQLRMATGLRNIPSIQSRG

SEQ ID NO: 3 - protein, 501 aa, Artificial Sequence
  HA1-C-CPE with N-terminal His-tag and secretion signal
MNFGLRLIFLVLTLKGVQAMGHHHHHHHHHENLYFQGGGGSGMDTICVGYHANNSTDTVDTILEKNVTVTHSVNLLENSHNGKLCSLNGKIPLQLGNCNVAGWILGNPKCDLLLTANSWSYIIETSNSKNGACYPGEFADYEELKEQLSTVSSFERFEIFPKATSWPNHDTTRGTTVACSHSGANSFYRNLLWIVKKGNSYPKLSKSYTNNKGKEVLVIWGVHHPPTDSDQQTLYQNNHTYVSVGSSKYYKRFTPEIVARPKVREQAGRMNYYWTLLDQGDTITFEATGNLIAPWHAFALKKGSSSGIMRSDAQVHNCTTKCQTPHGALKGNLPFQNVHPVTIGKCPKYVKSTQLRMATGLRNIPSIQSRGGGGSDIEKEILDLAAATERLNLTDALNSNPAGNLYDWRSSNSYPWTQKLNLHLTITATGQKYRILASKIVDFNIYSNNFNNLVKLEQSLGDGVKDHYVDISLDAGQYVLVMKANSSYSGNYPYSILFQKF

SEQ ID NO: 4 - protein, 459 aa, Artificial Sequence
  SITE 1..1: optionally present
  HA1-C-CPE without His-tag and without secretion signal
MDTICVGYHANNSTDTVDTILEKNVTVTHSVNLLENSHNGKLCSLNGKIPLQLGNCNVAGWILGNPKCDLLLTANSWSYIIETSNSKNGACYPGEFADYEELKEQLSTVSSFERFEIFPKATSWPNHDTTRGTTVACSHSGANSFYRNLLWIVKKGNSYPKLSKSYTNNKGKEVLVIWGVHHPPTDSDQQTLYQNNHTYVSVGSSKYYKRFTPEIVARPKVREQAGRMNYYWTLLDQGDTITFEATGNLIAPWHAFALKKGSSSGIMRSDAQVHNCTTKCQTPHGALKGNLPFQNVHPVTIGKCPKYVKSTQLRMATGLRNIPSIQSRGGGGSDIEKEILDLAAATERLNLTDALNSNPAGNLYDWRSSNSYPWTQKLNLHLTITATGQKYRILASKIVDFNIYSNNFNNLVKLEQSLGDGVKDHYVDISLDAGQYVLVMKANSSYSGNYPYSILFQKF

SEQ ID NO: 5 - protein, 263 aa, Artificial Sequence
  SARS-CoV-2 Spike S1 RBD with C-terminal His-tag and secretion signal
MNFGLRLIFLVLTLKGVQAMRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFVLVPRGSAAALEHHHHHHHH

SEQ ID NO: 6 - protein, 224 aa, SARS coronavirus
  SITE 1..1: optionally present
  SARS-CoV-2 Spike S1 RBD
MRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 7 - protein, 397 aa, Artificial Sequence
  SARS-CoV-2 Spike S1 RBD-C-CPE with N-terminal His-tag and secretion signal
MNFGLRLIFLVLTLKGVQAMGHHHHHHHHHENLYFQGGGGSGMRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFGGGGSDIEKEILDLAAATERLNLTDALNSNPAGNLYDWRSSNSYPWTQKLNLHLTITATGQKYRILASKIVDFNIYSNNFNNLVKLEQSLGDGVKDHYVDISLDAGQYVLVMKANSSYSGNYPYSILFQKF

SEQ ID NO: 8 - protein, 355 aa, Artificial Sequence
  SITE 1..1: optionally present
  RBD-C-CPE without His-tag and without secretion signal
MRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFGGGGSDIEKEILDLAAATERLNLTDALNSNPAGNLYDWRSSNSYPWTQKLNLHLTITATGQKYRILASKIVDFNIYSNNFNNLVKLEQSLGDGVKDHYVDISLDAGQYVLVMKANSSYSGNYPYSILFQKF

SEQ ID NO: 9 - protein, 1294 aa, Artificial Sequence
  Stabilized Spike trimer with C-terminal His/Strep-tag including trimerization domain
MALPVTALLLPLALLLHAARPMVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGASVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKGSGYIPEAPRDGQAYVRKDGEWVLLSTFLGENLYFQGGSAWSHPQFEKGGGSGGGSGGSAWSHPQFEKGSHHHHHHHH

SEQ ID NO: 10 - protein, 1224 aa, Artificial Sequence
  SITE 1..1: optionally present
  Stabilized Spike trimer including trimerization domain
VNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFMTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGASVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKGSGYIPEAPRDGQAYVRKDGEWVLLSTFL

SEQ ID NO: 11 - protein, 1396 aa, Artificial Sequence
  Stabilized Spike-C-CPE trimer with N-terminal His-tag, C-terminal trimerization domain and secretion signal
MNFGLRLIFLVLTLKGVQAMGHHHHHHHHHENLYFQGGGGSGMVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGASVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKGSGYIPEAPRDGQAYVRKDGEWVLLSTFLGGGSDIEKEILDLAAATERLNLTDALNSNPAGNLYDWRSSNSYPWTQKLNLHLTITATGQKYRILASKIVDFNIYSNNFNNLVKLEQSLGDGVKDHYVDISLDAGQYVLVMKANSSYSGNYPYSILFQKF

SEQ ID NO: 12 - protein, 1354 aa, Artificial Sequence
  SITE 1..1: optionally present
  Stabilized Spike-C-CPE trimer with trimerization domain
MVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGASVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKGSGYIPEAPRDGQAYVRKDGEWVLLSTFLGGGSDIEKEILDLAAATERLNLTDALNSNPAGNLYDWRSSNSYPWTQKLNLHLTITATGQKYRILASKIVDFNIYSNNFNNLVKLEQSLGDGVKDHYVDISLDAGQYVLVMKANSSYSGNYPYSILFQKF

SEQ ID NO: 13 - protein, 1367 aa, Artificial Sequence
  Stabilized Spike-C-CPE monomer with N-terminal His-tag and secretion signal
MNFGLRLIFLVLTLKGVQAMGHHHHHHHHHENLYFQGGGGSGMVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGASVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKGGGSDIEKEILDLAAATERLNLTDALNSNPAGNLYDWRSSNSYPWTQKLNLHLTITATGQKYRILASKIVDFNIYSNNFNNLVKLEQSLGDGVKDHYVDISLDAGQYVLVMKANSSYSGNYPYSILFQKF

SEQ ID NO: 14 - protein, 1325 aa, Artificial Sequence
  SITE 1..1: optionally present
  Stabilized Spike-C-CPE monomer
MVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGASVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKGGGSDIEKEILDLAAATERLNLTDALNSNPAGNLYDWRSSNSYPWTQKLNLHLTITATGQKYRILASKIVDFNIYSNNFNNLVKLEQSLGDGVKDHYVDISLDAGQYVLVMKANSSYSGNYPYSILFQKF